SEQUENCE LISTING (50 sequences)

SEQ ID NO: 1 - DNA, 20 bp, Artificial Sequence
  Oligonucleotide
gctctcaccctcctctcctg

SEQ ID NO: 2 - DNA, 20 bp, Artificial Sequence
  Oligonucleotide
tatcctattctcctccttgg

SEQ ID NO: 3 - DNA, 24 bp, Artificial Sequence
  Oligonucleotide
ctccagctttgtgctctgcctctg

SEQ ID NO: 4 - DNA, 23 bp, Artificial Sequence
  Oligonucleotide
agtacaacaaggccaccgaagat

SEQ ID NO: 5 - DNA, 23 bp, Artificial Sequence
  Oligonucleotide
agaagcaagaaggaaggagggag

SEQ ID NO: 6 - DNA, 23 bp, Artificial Sequence
  Oligonucleotide
tacaacaaggccaccgaagatga

SEQ ID NO: 7 - DNA, 23 bp, Artificial Sequence
  Oligonucleotide
agaagcaagaaggaaggagggag

SEQ ID NO: 8 - DNA, 23 bp, Artificial Sequence
  Oligonucleotide
tgctactcctgatggctaccctg

SEQ ID NO: 9 - DNA, 23 bp, Artificial Sequence
  Oligonucleotide
gtggccttgttgtactcgctgat

SEQ ID NO: 10 - DNA, 23 bp, Artificial Sequence
  Oligonucleotide
agtacaacaaggccaccgaagat

SEQ ID NO: 11 - DNA, 27 bp, Artificial Sequence
  Oligonucleotide
taccaggtctattagaagcaagaagga

SEQ ID NO: 12 - DNA, 23 bp, Artificial Sequence
  Oligonucleotide
tgctactcctgatggctaccctg

SEQ ID NO: 13 - DNA, 23 bp, Artificial Sequence
  Oligonucleotide
catcttcggtggccttgttgtac

SEQ ID NO: 14 - DNA, 23 bp, Artificial Sequence
  Oligonucleotide
tgctactcctgatggctaccctg

SEQ ID NO: 15 - DNA, 23 bp, Artificial Sequence
  Oligonucleotide
tactcatcttcggtggccttgtt

SEQ ID NO: 16 - DNA, 25 bp, Artificial Sequence
  Oligonucleotide
tgggattatcctattctcctccttg

SEQ ID NO: 17 - DNA, 23 bp, Artificial Sequence
  Oligonucleotide
ctccagctttgtgctctgcctct

SEQ ID NO: 18 - DNA, 23 bp, Artificial Sequence
  Oligonucleotide
tgctactcctgatggctaccctg

SEQ ID NO: 19 - DNA, 23 bp, Artificial Sequence
  Oligonucleotide
ctcatcttcggtggccttgttgt

SEQ ID NO: 20 - DNA, 23 bp, Artificial Sequence
  Oligonucleotide
tacagtgggtgggagtgggtggt

SEQ ID NO: 21 - DNA, 23 bp, Artificial Sequence
  Oligonucleotide
gagtgggtacagcgtgcccttca

SEQ ID NO: 22 - DNA, 24 bp, Artificial Sequence
  Oligonucleotide
tgcctcgggctctcaccctcctct

SEQ ID NO: 23 - DNA, 24 bp, Artificial Sequence
  Oligonucleotide
tgggtggtggtcggtgtgactggc

SEQ ID NO: 24 - DNA, 19 bp, Artificial Sequence
  Oligonucleotide
agtcccagcccaacttgga

SEQ ID NO: 25 - DNA, 24 bp, Artificial Sequence
  Oligonucleotide
gggaacttcgtagatctggaaaga

SEQ ID NO: 26 - DNA, 20 bp, Artificial Sequence
  Oligonucleotide
cagaagaaacagttgtgctc

SEQ ID NO: 27 - DNA, 18 bp, Artificial Sequence
  Oligonucleotide
ggagtaggaggtggtcag

SEQ ID NO: 28 - DNA, 18 bp, Artificial Sequence
  Oligonucleotide
tctcaccctcctctcctg

SEQ ID NO: 29 - DNA, 21 bp, Artificial Sequence
  Oligonucleotide
ttatcctatcctcctccttgg

SEQ ID NO: 30 - DNA, 19 bp, Artificial Sequence
  Oligonucleotide
aagatcattgctcctcctg

SEQ ID NO: 31 - DNA, 18 bp, Artificial Sequence
  Oligonucleotide
cgtcatactcctgcttgc

SEQ ID NO: 32 - DNA, 45 bp, Artificial Sequence
  Oligonucleotide
aattctaatacgactcactataggggctctcaccctcctctcctg

SEQ ID NO: 33 - DNA, 25 bp, Artificial Sequence
  Oligonucleotide
gggctctggcctcgagctccaagga

SEQ ID NO: 34 - DNA, 30 bp, Artificial Sequence
  Oligonucleotide
ataggataatcccaggtggcatctatgatg

SEQ ID NO: 35 - DNA, 30 bp, Artificial Sequence
  Oligonucleotide
tctcctccttggagctcgaggccagagccc

SEQ ID NO: 36 - DNA, 30 bp, Artificial Sequence
  Oligonucleotide
catcatagatgccacctgggattatcctat

SEQ ID NO: 37 - DNA, 23 bp, Artificial Sequence
  Oligonucleotide
cccggcctctgtgtaccctgcta

SEQ ID NO: 38 - DNA, 30 bp, Artificial Sequence
  Oligonucleotide
gaactcgagctaccctggctggggctctgg

SEQ ID NO: 39 - DNA, 24 bp, Artificial Sequence
  Oligonucleotide
ggtggccttgttgtactcgctgat

SEQ ID NO: 40 - DNA, 28 bp, Artificial Sequence
  Oligonucleotide
gctctcgagagtgaagggcacgctgtac

SEQ ID NO: 41 - DNA, 29 bp, Artificial Sequence
  Oligonucleotide
ttactcgagctccaaggaggagaatagga

SEQ ID NO: 42 - DNA, 736 bp, Homo sapiens
ggctctcaccctcctctcctgcagctccagctttgtgctctgcctctgaggagaccatggcccggcctctgtgtaccctgctactcctgatggctaccctggctggggctctggcctcgagctccaaggaggagaataggataatcccaggtggcatctatgatgcagacctcaatgatgagtgggtacagcgtgcccttcacttcgccatcagcgagtacaacaaggccaccgaagatgagtactacagacgcccgctgcaggtgctgcgagccagggagcagacctttgggggggtgaattacttcttcgacgtagaggtgggccgcaccatatgtaccaagtcccagcccaacttggacacctgtgccttccatgaacagccagaactgcagaagaaacagttgtgctctttcgagatctacgaagttccctgggaggacagaatgtccctggtgaattccaggtgtcaagaagcctaggggtctgtgccaggccagtcacaccgaccaccacccactcccacccactgtagtgctcccacccctggactggtggcccccaccctgcgggaggcctccccatgtgcctgtgccaagagacagacagagaaggctgcaggagtcctttgttgctcagcagggcgctctgccctccctccttccttcttgcttctaatagacctggtacatggtacacacacccccacctcctgcaattaaacagtagcatcgcc

SEQ ID NO: 43 - DNA, 142 bp, Homo sapiens
gctctcaccctcctctcctgcagctccagctttgtgctctgcctctgaggagaccatggcccggcctctgtgtaccctgctactcctgatggctaccctggctggggctctggcctcgagctccaaggaggagaataggata

SEQ ID NO: 44 - DNA, 759 bp, Homo sapiens
gggctccctgcctcgggctctcaccctcctctcctgcagctccagctttgtgctctgcctctgaggagaccatggcccggcctctgtgtaccctgctactcctgatggctaccctggctggggctctggcctcgagctccaaggaggagaataggataatcccaggtggcatctatgatgcagacctcaatgatgagtgggtacagcgtgcccttcacttcgccatcagcgagtacaacaaggccaccgaagatgagtactacagacgcccgctgcaggtgctgcgagccagggagcagacctttgggggggtgaattacttcttcgacgtagaggtgggccgcaccatatgtaccaagtcccagcccaacttggacacctgtgccttccatgaacagccagaactgcagaagaaacagttgtgctctttcgagatctacgaagttccctgggaggacagaatgtccctggtgaattccaggtgtcaagaagcctaggggtctgtgccaggccagtcacaccgaccaccacccactcccacccactgtagtgctcccacccctggactggtggcccccaccctgcgggaggcctccccatgtgcctgtgccaagagacagacagagaaggctgcaggagtcctttgttgctcagcagggcgctctgccctccctccttccttcttgcttctaatagacctggtacatggtacacacacccccacctcctgcaattaaacagtagcatcgcctccctctg

SEQ ID NO: 45 - DNA, 284 bp, Homo sapiens
ggctctcaccctcctctcctgcagctccagctttgtgctctgcctctgaggagaccatggcccggcctctgtgtaccctgctactcctgatggctaccctggctggggctctggcctcgagctccaaggaggagaataggataatcccaggtggcatctatgatgcagacctcaatgatgagtgggtacagcgtgcccttcacttcgccatcagcgagtacaacaaggccaccgaagatgagtactacagacgcccgctgcaggtgctgcgagccagggagcag

SEQ ID NO: 46 - DNA, 114 bp, Homo sapiens
acctttgggggggtgaattacttcttcgacgtagaggtgggccgcaccatatgtaccaagtcccagcccaacttggacacctgtgccttccatgaacagccagaactgcagaag

SEQ ID NO: 47 - DNA, 338 bp, Homo sapiens
aaacagttgtgctctttcgagatctacgaagttccctgggaggacagaatgtccctggtgaattccaggtgtcaagaagcctaggggtctgtgccaggccagtcacaccgaccaccacccactcccacccactgtagtgctcccacccctggactggtggcccccaccctgcgggaggcctccccatgtgcctgtgccaagagacagacagagaaggctgcaggagtcctttgttgctcagcagggcgctctgccctccctccttccttcttgcttctaatagacctggtacatggtacacacacccccacctcctgcaattaaacagtagcatcgcc

SEQ ID NO: 48 - protein, 141 aa, Homo sapiens
MARPLCTLLLLMATLAGALASSSKEENRIIPGGIYDADLNDEWVQRALHFAISEYNKATEDEYYRRPLQVLRAREQTFGGVNYFFDVEVGRTICTKSQPNLDTCAFHEQPELQKKQLCSFEIYEVPWEDRMSLVNSRCQEA

SEQ ID NO: 49 - DNA, 426 bp, Homo sapiens
atggcccggcctctgtgtaccctgctactcctgatggctaccctggctggggctctggcctcgagctccaaggaggagaataggataatcccaggtggcatctatgatgcagacctcaatgatgagtgggtacagcgtgcccttcacttcgccatcagcgagtacaacaaggccaccgaagatgagtactacagacgcccgctgcaggtgctgcgagccagggagcagacctttgggggggtgaattacttcttcgacgtagaggtgggccgcaccatatgtaccaagtcccagcccaacttggacacctgtgccttccatgaacagccagaactgcagaagaaacagttgtgctctttcgagatctacgaagttccctgggaggacagaatgtccctggtgaattccaggtgtcaagaagcctag

SEQ ID NO: 50 - protein, 121 aa, Homo sapiens
SSSKEENRIIPGGIYDADLNDEWVQRALHFAISEYNKATEDEYYRRPLQVLRAREQTFGGVNYFFDVEVGRTICTKSQPNLDTCAFHEQPELQKKQLCSFEIYEVPWEDRMSLVNSRCQEA

(19) United States
(12) Patent Application Publication
(10) Pub. No.: US 2015/0168410 A1
(43) Pub. Date: Jun. 18, 2015

(54) BIOMARKERS FOR COLORECTAL CANCER DIAGNOSIS AND PREDICTION

(71) Applicants: Tao Wang, Suzhou (CN); Xiangyun Qu, Suzhou (CN); Fei Chen, Suzhou (CN)

(72) Inventors: Tao Wang, Suzhou (CN); Xiangyun Qu, Suzhou (CN); Fei Chen, Suzhou (CN)

(73) Assignee: Suzhou MicroDiag Biomedicine Co., Ltd, Suzhou, Jiangsu (CN)

(21) Appl. No.: 14/371,088

(22) PCT Filed: Jan. 9, 2012
(86) PCT No.: PCT/CN2012/070149

§ 371 Date: Dec. 8, 2014

(51) Int. Cl. G01N 33/574; C07K 16/38; C12Q 1/68

(52) U.S. Cl. CPC G01N 33/57419; C12Q 1/6886; C07K 16/38; C12Q 2600/158; G01N 2333/8139

(57) ABSTRACT

In this invention, we disclosed and documented the applications of CST4 gene, mRNA of CST4, cDNA of CST4 splices, the corresponding amplicon of CST4-specific primers, cystatin S protein coded by CST4 gene and epitope peptide of CST4 in the diagnosis and prediction of colorectal cancer, and the detection method and testing kits of the above. This invention shall be applied in the diagnosis, dynamic monitoring and progression prediction of colorectal cancer. Methods mentioned in this invention features high sensitivity and reliability, which was verified by large-scale trial.

### TECHNOLOGICAL FIELD

This invention is of biotechnology and biomedicine. Particularly this invention discloses biomarkers for colorectal cancer and their applications such as the diagnosis, dynamic monitoring and progression prediction of colorectal cancer, the testing reagents and kits and their protocols.

### BACKGROUND

The early detection and treatment for colorectal cancer have improved much recently. However, around 540 thousand deaths due to colorectal cancer were reported in 2008. The domestic mortality rate of colorectal cancer increases by 3% annually. Many problems for the prevention of colorectal cancer remain to be solved, such as early detection, timely intervention, treatment efficacy assessment and monitoring and for post-treatment patients the real time warning of cancer recurrence with high accuracy.

It is critical for improve the early detection rate and progression prediction accuracy of colorectal cancer to develop diagnostic tool for colorectal cancer with high sensitivity and specificity. Cystatin superfamily plays important roles in the occurrence and development of tumors as cathepsin inhibitors. Proteins belonging to the superfamily reversibly bind cysteine protease in tissue and body fluids to prevent the over-activity of cathepsin. Cystatin C is the strongest inhibitor for cathepsin B ever. The expression of cystatin C increases in ovarian cancer and head and neck cancer patients. The expression of stefin A, another member of the cystatin superfamily, increases with the occurrence of non-small cell lung cancer (NSCLC). The mRNA expression of stefin B, however, is lower than normal for meningiomas patients. Increased expression of cystatin F (or lekocystatin and CMPA) is observed on cancer patients too. It is hypothesized or reported that the occurrence and development of tumors require the participation of cathepsin, whose expression is increased. The expression of cystatin is then increase as a result due to the feedback mechanism is activated to inhibit the over-activity of the protease. It should be noted that cystatin expression does not always correlate with the progression of tumors. For instance, for glioma patients, lower cystatin C expression (both protein and mRNA) is correlated with shorter survival time and higher probability of recurrence.

In this invention, we verified the strong correlation of CST4, a member of the cystatin superfamily and its splices with colorectal cancer. CST4, or cystatin S, is a cysteine protease inhibitor with 141 amino acid residues. It is found in multiple body fluids and secretions such as tear, saliva, plasma and serum.

### SUMMARY OF THE INVENTION

One of the goals of this invention is to provide the novel applications of CST4 gene, mRNA of CST4, cDNA of CST4 splices, amplicon of CST4-specific primers, cystatin S protein and its epitopes as a new tool for colorectal cancer diagnosis.

1. To achieve the goal, the technology of this invention is described as following. CST4 gene, mRNA of CST4, cDNA of CST4 splices, amplicon of CST4-specific primers, cystatin S protein and its epitopes as are applied as biomarkers for colorectal cancer diagnosis and prediction. The sequence of CST4 gene is shown in SEQ ID No.42.

The preferred sequence for the probe for the detections of CST4 gene, mRNA of CST4, and cDNA of CST4 splices is presented in SEQ ID No.3.

Favorably, the specific primers of the amplicon have sequences shown in SEQ ID No.1, 4, 6, 8, 10, 12, 14, 16, 18, 20 (primer 1) and in SEQ ID No.2, 5, 7, 9, 11, 13, 15, 17, 19, 21 (primer 2). Sequence in SEQ ID No.1 pairs with sequence in SEQ ID No.2. Sequence in SEQ ID No.4 pairs with sequence in SEQ ID No.5. Sequence in SEQ ID No.6 pairs with sequence in SEQ ID No.7. Sequence in SEQ ID No.8 pairs with sequence in SEQ ID No.9. Sequence in SEQ ID No.10 pairs with sequence in SEQ ID No.11. Sequence in SEQ ID No.12 pairs with sequence in SEQ ID No.13. Sequence in SEQ ID No.14 pairs with sequence in SEQ ID No.15. Sequence in SEQ ID No.16 pairs with sequence in SEQ ID No.17. Sequence in SEQ ID No.18 pairs with sequence in SEQ ID No.19. Sequence in SEQ ID No.20 pairs with sequence in SEQ ID No.21.

The preferred sequence for cystatin S epitope peptide is presented in SEQ ID No.50.

Diagnosis and prediction is favorably defined as the metastasis, micro-metastasis, pTNM stage determination, dynamic monitoring during treatment and progression prediction of colorectal cancer.

A second objective of this invention is to provide several capturers with specific affinity to the colorectal cancer biomarkers.

The technological details for the realization of the objective mentioned above are described as following.

Capturers for colorectal cancer biomarkers are for the diagnosis and prediction of colorectal cancers. The colorectal cancer biomarkers are CST4 gene, mRNA of CST4, cDNA of CST4 splices, amplicon of CST4-specific primers, cystatin S coded by CST4 and its epitope peptides.

Sequences of the CST4 primers (mentioned in 3) are shown in SEQ ID No.1-2.

Sequence of the probe is presented in SEQ ID No.3.

The sequence of the amplicon (mentioned in 3) is shown in SEQ ID No.43.

The capturers mentioned are antibodies that specifically recognize cystatin S or its epitopes.

The sequence of cystatin S epitope peptide is shown in SEQ ID No.50.

Another objective is to provide novel applications for the capturers. Testing kits and protocols based on these applications should be provided, too. The applications and testing kits are new for the detections of colorectal cancer with high accuracy, simple operation and suitability for extensive clinical practice.

The technical details for the realization of the objective are described as following.

This section includes the applications of the capturers in the testing reagents and kits for colorectal cancer detections and diagnostic kits that include the capturers mentioned above.

The testing kits are described as following.

1) testing kits for quantitative and real time detection of the mRNA of CST4 based on hydrolytic Taqman probe. The primers sequences are presented in SEQ ID No.1-2; the probe sequence is presented in SEQ ID No.3.

2) Testing kits for quantitative and real time detection of the mRNA of CST4 based on fluorescent dye. The primers sequences are presented in SEQ ID No.1-2. Sequences for internal calibration primers are shown in SEQ ID No.30-31.

3) Testing kits for quantitative and real time detection of the mRNA of CST4 based on nucleic acid based amplification (NASBA) or transcription-median amplification (TMA). Both kits include primers and probes for CST4, whose sequences are shown in SEQ ID No.2, 32 (for primers) and 3 (for probe).

4) Testing kits for quantitative and real time detection of the mRNA of CST4 based on ligase chain reaction (LCR). Four probes are included whose sequences are shown in SEQ ID No.33-36.

5) Testing kits for quantitative and real time detection of the mRNA of CST4 based on thermophilic strand displacement amplification (tSDA). Primers (sequences shown in SEQ ID No.37-40) and a probe (SEQ ID No. 41) are included.

The diagnostic kits are preferred as following.

1) Double-antibody sandwich ELISA kits, including the solid substrate, capturers immobilized on the solid substrate, biotinylated capturers and the enzymatic substrate (colorimetric). Capturers immobilized are monoclonal antibodies while biotinylated capturers are polyclonal antibodies.

2) Blotting kits including solid substrate, capturers, enzymatic labeled secondary antibody and enzymatic substrate for colorimetric detections. The capturers are monoclonal antibodies and biotinylated capturers are polyclonal antibodies.

3) Competitive ELISA kits including solid substrate, immobilized antigen, biotinylated capturers, the enzymatic substrate for colorimetric detections and specific monoclonal antibody. The biotinylated capturers are polyclonal antibodies.

All diagnostic kits mentioned above include positive and negative control samples and blank sample.

The testing kit based on double-antibody sandwich ELISA uses rat-anti-cystatin S monoclonal antibody as the specific antibody. The solid substrate is the ELISA plate. The biotinylated specific polyclonal antibody is biotinylated rabbit-anti-cystatin S polyclonal antibody.

The solid substrate of the testing kit based on double-antibody sandwich ELISA is an ELISA plate. The capturer immobilized on the solid substrate is rat-anti-cystatin S monoclonal antibody (R&D, MAB 1296, 5 μg/mL). The biotinylated capturer is rabbit-anti-cystatin S polyclonal antibody (with valence of 1:1000). The substrate for colorimetric detection is alkaline phosphatase.

The solid substrate of the testing kit based on competitive ELISA is an ELISA plate. The concentration of cystatin S is 5 μg/mL. The specific monoclonal antibody is rat-anti-cystatin S monoclonal antibody (R&D, MAB 1296, with valence of 1:2000). The enzymatic labeled secondary antibody is ALP labeled goat-anti-mouse IgG (Jackson, dissolved in TBS with 3% BSA, with valence of 1:2000). The enzymatic substrate is ALP substrate. Volume ratio of cystatin S, enzymatic labeled secondary antibody and enzymatic substrate is 1:2.

The solid substrate of the testing kit based on immune-blotting is nitrocellulose membrane. The capturer is rat-anti-cystatin S (with valence of 1:1000) antibody. The enzymatic labeled secondary antibody is peroxidase labeled goat-anti-rabbit IgG (Jackson). The enzymatic substrate is commercial TMB solution (Kirkegaard and Perry Laboratories Inc., Gaithersburg, Md., “TMB peroxidase substrate solution” Cat. No. 50-76-01).

The protocols of the testing kits are described as following in detail.

The ELISA plate (Corning) is coated with cystatin S (Abnova Cat. No. H00001472-P01) solution (5 ng/mL) and backfilled with 3% BSA solution. Rat-anti-cystatin S monoclonal antibody (R&D Cat. No. MAB 1296, with valence of 1:2000) and serum (with 8× dilution) are mixed and incubated with the plate under 4° C. overnight, followed by incubation of the mixture in the plate under 37° C. for an hour. Holes of the plates are then washed by TBS buffer (10 mM Tris-HCl, 154 mM NaCl, pH 7.5). ALP-labeled goat-anti-mouse IgG (Jackson, with valence of 1:2000, dissolved in TBS buffer with 0.3% BSA) is added to the plate and incubated under 37° C. for an hour. ALP substrate (Kirkegaard and Perry Laboratories Inc., Gaithersburg, Md., blue Phos Solution Cat. No. 508805, 100 μL per hole). OD vale is finally read using a microplate reader.

The fourth objective is to provide in vitro diagnostic tool and kits with easy operation, high sensitivity and specificity.

The detailed description of the technology to realize the aim is as following.

Colorectal cancer can be detected or predicted by the diagnostic kits mentioned above. In more detailed words, the expression level or the quantitative content of the colorectal cancer markers measured via the testing kits is compared with those of healthy subjects to decide whether the result is positive or not. Or the result is read positive if it is higher than a constant (cutoff value). The cutoff value is obtained through the comparison of the colorectal cancer markers expressions/levels in the body fluids or tissue samples of colorectal cancer patients and healthy subjects. The cutoff value is of statistical significance. Samples include one or more of the following: blood, urine, marrow, colorectal cancer cell lines, colorecatal cancer tumors and tumor adjacent tissues and lymph node tissues. For instance, the cutoff value of cystatin S in this case is 3.434 ng/mL.

The testing kits for the prediction and diagnosis of colorectal cancer are kits for the measurements of cystatin S protein expression. The kits include solid substrate, capturers immobilized on the solid substrate, biotinylated capturers and enzymatic substrate for colorimetric detections. The capturers immobilized are monoclonal antibodies and the biotinylated capturers are polyclonal antibodies.

Or the testing kits for cystatin S protein expression measurement include solid substrate, cystatin S protein coated on the plate, rat-anti-cystatin S monoclonal antibodies, enzymatic labeled secondary antibodies and enzymatic substrate for colorimetric detections.

Or the testing kits for cystatin S protein expression measurement include solid substrate, capturers, enzymatic labeled secondary antibodies and enzymatic substrate for colorimetric detections. Capturers are monoclonal antibodies and biotinylated capturers are polyclonal antibodies.

Testing kits are based on double-antibody sandwich ELISA, wherein the solid substrate of the assay is the ELISA plate; capturers immobilized are rat-anti-cystatin S monoclonal antibody; the biotinylated capturers are rabbit-anti-cystatin S polyclonal antibody with valence of 1:1000. The enzymatic substrate is ALP.

Or the kits are based on competitive ELISA, wherein the solid substrate of the assay is the ELISA plate; the concentration of cystatin S is 5 μg/mL; the monoclonal antibody is rat-anti-cystatin S antibody with valence of 1:2000; the enzymatic labeled secondary antibodies are ALP labeled goat-anti-mouse IgG with valence of 1:2000. The enzymatic substrate is ALP. The volume ratio of cystatin S, enzymatic labeled secondary antibody and the substrate is 1:2.

Or the testing kits based on immunoblotting, wherein the solid substrate is the nitrocellulose membrane; the capturers are rat-anti-cystatin S monoclonal antibodies with valence of 1:1000; the enzymatic labeled secondary antibodies are HRP-labeled goat-anti-rabbit IgG; the enzymatic substrate for colorimetric detections are TMB solution.

Advantages of this invention are 1) the applications of the expression CST4 gene and cystatin S protein in the diagnosis of colorectal cancers, real time monitoring of the cancer development and colorectal cancer progression prediction are verified in this invention with large-scale trial. The results are of significant accuracy; the invention can be applied in the development of novel methods for colorectal cancer diagnosis and real time monitoring, as well as colorectal cancer prognosis predictions; 2) Testing reagents and kits with high sensitivity for colorectal cancer diagnosis and real time monitoring, as well as colorectal cancer progression predictions, are included in this invention.

### BRIEF DESCRIPTION OF THE FIGURES

FIG. 1. Recombinant plasmid Pmd18-T that involve CST4

FIG. 2. Expression (measured by biochips) of CST4 gene in normal tissues including tonsil, posterior pituitary, thyroid, salivary gland, skeletal muscle, bone marrow, peripheral blood without red blood cells and platelets, lung, stomach, liver, heart, kidney, adrenal gland, intestines, colon, pancreas, spleen, bladder, prostate, ovarian, uterus, placenta and testis), human colorectal cancer cell lines SW480 and caco2 and human intestinal epithelial cell line HNIEC.

FIG. 3. Expression comparison of CST124, CST1, CST2, CST4 in 20 cases of colorectal cancer tumors and their respective tumor adjacent tissues using real time qPCR with fluorescent dye as the probe.

FIG. 4. Expression of CST4 quantified by real time PCR in 100 cases of colorectal cancer tumors and their respective tumor adjacent tissues.

FIG. 5. Expression of CST4 quantified by real time PCR in 36 colorectal cancer samples and 29 colonospcopic specimens with enteritis

FIG. 6. Expression of CST4 quantified by real time PCR in 20 lymph node samples with colorectal cancer metastasis and 15 lymph node samples without colorectal cancer metastasis. Metastasis was verified by pathological studies.

FIG. 7. Comparison of accuracies for colorectal cancer diagnosis using cytological studies and CST4 expression in peripheral blood quantified by real time PCR.

FIG. 8. Comparison of accuracies for colorectal cancer marrow metastasis predictions using cytological studies and CST4 expression quantified by real time PCR.

FIG. 9. CST4 expression of serum cell-free RNA of 50 colorectal cancer patients, 30 healthy subjects and 30 enteritis patients quantified by real time PCR (A) and receiver operating characteristic (ROC) curve (B). ROC curve can be used for the evaluation of the sensitivity and specificity of the method to distinguish colorectal cancer patients from enteritis patients and healthy subjects.

FIG. 10. CST4 expression of serum cell-free RNA of 50 colorectal cancer patients, 30 healthy people and 30 enteritis patients quantified by ligase chain reaction (LCR).

FIG. 11. CST4 expression of cell-free RNA in serums of 50 colorectal cancer patients, 30 healthy subjects and 30 enteritis patients quantified by reverse transcription strand displacement amplification (rtSDA).

FIG. 12. CST4 expression in urine samples of 20 colorectal cancer patients, 10 healthy people and 10 enteritis patients quantified by nucleic acid based amplification (NASBA).

FIG. 13. CST4 expression by transcription-mediated amplification (TMA) of 80 colorectal patients with various pTNM stages (30 I+II cases and 50 III+IV cases).

FIG. 14. Cystatin S expression in culture supernatant of colorectal cancer cell lines and serums of healthy subjects.

FIG. 15. Cystatin S expression in serums of colorectal cancer and healthy people.

FIG. 16. Cystatin S expression by competitive ELISA in serum samples of 30 colorectal cancer patients and 20 healthy people.

FIG. 17. Specificity and sensitivity comparison of cystatin S and CEA (measured by ELISA) for colorectal cancer detection.

FIG. 18. Prognosis-free survival (PFS) curve of post-treatment colorectal cancer patients with higher or lower cystatin S expression than the median value.

### DETAILED DESCRIPTION OF THE INVENTION

### Part I Molecular Detection

The molecular biological technologies mentioned above are illustrated by the following examples. It should be noted that these example are for clarification for this invention instead of limiting the applications of this invention. Protocols in Molecular Cloning: A Laboratory Manual (Edited by J. Sambrook et al) were strictly followed for experimental procedures if not noted. Or guidelines from the manufacturers were followed. If not mentioned, percentage and fraction are based on weight.

### Materials and Methods

All clinical samples were acquired from Beijing Friendship Hospital, with the regulations of the hospital strictly observed and consent forms signed by the patients.

Biopsy samples suspected as tumor or their adjacent tissues were compared. RNA in these samples as well as the lymph node samples should be immediately extracted after sample acquisition or they should be stored in liquid nitrogen or RNAlater (Ambion).

Peripheral blood, marrow or urine samples were centrifuged for 20 minutes (4000 rpm, 4° C.). The supernatant is centrifuged for another 10 minutes (13000 rpm, 4° C.). Separate the supernatant and precipitation. RNA extraction should follow immediately or the samples be stored under −20° C. or −80° C.

Real Time PCR Using TaqMan Probes:

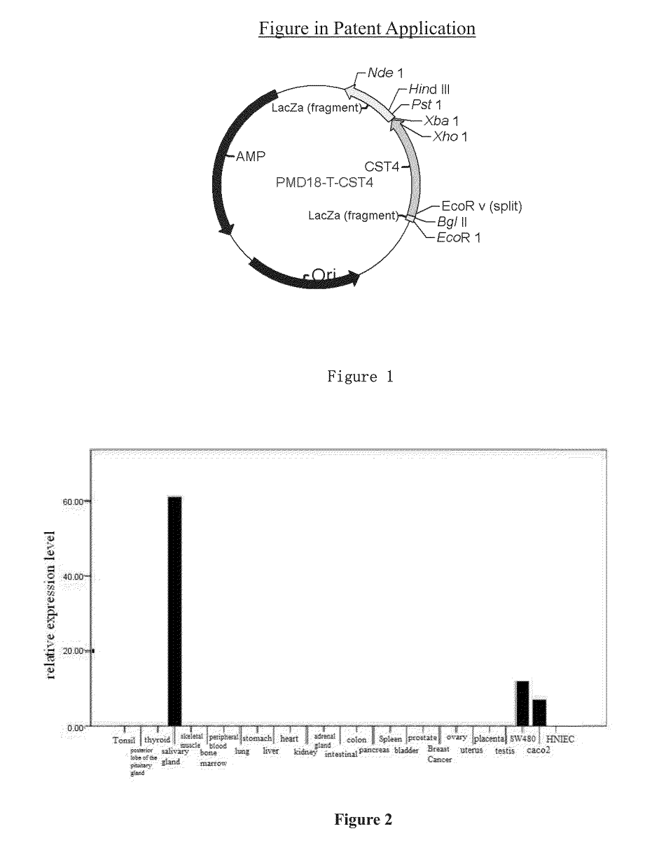

Samples were subjected to nucleic acid extraction using commercial kits. A non-limiting example is phenol-chloroform extraction. RNA in the samples was obtained following the Trizol kit manufactured by Invitrogen. The quality of the RNA extracted was examined following protocols in references such as Molecular Biology Experiments (by J. Li). Reverse transcription of mRNA was realized by commercially available kits and the guidelines were followed. cDNA solutions were prepared with appropriate concentration gradients. Primers for biochemical reactions were optimized. The primers for CST4 were designed based on exon 1. Recombinant plasmid with CST4 amplicon are commercially available from Pegm-T (Promega, as shown in FIG. 1). Primers were designed based on exons 1 and 3. The PCR process was monitored by the hydrolysis of the TaqMan probe.

The optimized sequences for primers of CST4 are: gctctcaccctcctctcctg (SEQ ID No.1) and tatcctattctcctccttgg (SEQ ID No.2). The sequence of the probe is 5′-fam-ctccagctttgtgctctgcctctg-tamra-3′ (SEQ ID No.3). The size of the amplicon is 142 bp.

The optimized sequences for primers of recombinant plasmid that contains CST4 amplicon are tgcctcgggctctcaccctcctct (SEQ ID No.22) and tgggtggtggtcggtgtgactggc (SEQ ID No.23).

In a typical testing, the sample, positive and negative controls and recombinant plasmid standards are amplified simultaneously. Cross points (CP) of the recombinant plasmid standards with appropriate concentration gradients versus concentration are plotted and a calibration curve is obtained. Copies of the gene expression in the sample and control sample are quantified through the curve.

Real Time PCR Using Fluorescent Dyes as the Probe:

Pre-treatments for the samples are identical to what is described in the section of real time PCR using TaqMan probes. The sequences for primers for the amplifications of CST1, CST2 and CST4, which are amplified simultaneously, are agtcccagcccaacttgga (SEQ ID No.24) and gggaacttcgtagatctggaaaga (SEQ ID No.25). The sequences of the primers for CST4 amplifications are agtacaacaaggccaccgaagat (SEQ ID No.4) and agaagcaagaaggaaggagggag (SEQ ID No. 5), or tacaacaaggccaccgaagatga (SEQ ID No.6) and agaagcaagaaggaaggagg gag (SEQ ID No.7), or tgctactcctgatggctaccctg (SEQ ID No.8) and gtggccttgttgtactcgctgat (SEQ ID No. 9), or agtacaacaaggccaccgaagat (SEQ ID No.10) and taccaggtctattagaagcaagaagga (SEQ ID No. 11), or tgctactcctgatggctaccctg (SEQ ID No.12) and catcttcggtggccttgttgtac (SEQ ID No. 13), or tgctactcctgatggctaccctg (SEQ ID No.14) and tactcatctt cggtggccttgtt (SEQ ID No. 15), or tgggattatcctattctcctccttg (SEQ ID No.16) and ctccagcttt gtgctctgcctct (SEQ ID No. 17), or tgctactcctgatggctaccctg (SEQ ID No.18) and ctcatcttcg gtggccttgt tgt (SEQ ID No. 19), or tacagtgggtgggagtgggtggt (SEQ ID No.20) and gagtgggtac agcgtgccct tca (SEQ ID No. 21). The sequences of the primers for CST2 amplifications are cagaagaaacagttgtgctc (SEQ ID No.26) and ggagtaggaggtggtcag (SEQ ID No.27). The sequences of the primers for CST1 amplifications are tctcaccctcctctcctg (SEQ ID No.28) and ttatcctatcctcctccttgg (SEQ ID No.29). β-actin is applied as an internal reference. The sequences of the primers for β-actin amplifications are aagatcattgctcctcctg (SEQ ID No.30) and cgtcatactcctgcttgc (SEQ ID No.31). Genes in cancerous tumors, tumor adjacent tissues (TAT) and internal reference gene are amplified simultaneously. The copies of the genes are quantified by the following equation, wherein ct is the cycle number when the fluorescence signal is over the background. SYBR Greenk, Eve Green, LC Green, etc are among fluorescent dyes that apply.

$${Expression}_{relative} = \frac{{2}^{{ct}_{tumor} - {ct}_{ref}}}{{2}^{{ct}_{TAT} - {ct}_{ref}}}$$

In Vitro RNA Amplification by Nucleic Acid Based Amplification (NASBA):

The kits include T7 RNA polymerase, RNase H, avian myeloid leukemia virus (AMV) reverse transcriptase, ribonucleotide triphosphate (NTP), deoxyribonucleotide triphosphate (dNTP), primers for CST4 amplifications which were mentioned in the section of “real time PCR using TaqMan probes”, fluorescent dyes for RNA amplification monitoring (Ribo-Green fluorescent dye). The RNA template is amplified by 2⁹-2¹² fold after 2 hours incubation under 42° C. The fluorescence of the amplified product was monitored for the quantification of the template concentration before the amplification.

### Example 1

### Expression of CST4 and Other Members of the CST Superfamily

1. CST4 in Various Human Tissues

All tissue samples were purchased except the colon tissues which were acquired from Beijing Friendship Hospital (BFH). CST4 mRNA expression in various human tissue samples was measured on HG-U95AV Human GeneChip Array (Affymetirx) and compared; protocols from the user manual were followed. Quantifications of CST1 mRNA expressions were realized by the β-actin fluorescence calibration curve. As shown in FIG. 2, CST4 expression is low in all samples except in salivary gland. No CST4 expression was observed in other tissues tested. The result indicates that CST4 is promising pathological diagnosis because of its low background signal. CST4 is over-expressed in SW480 and Caco2, which are colorectal cancer cell lines and not expressed in HNIEC, which is normal colon tissue cell lines. It is concluded that CST4 is a promising marker for colorectal cancer diagnosis.

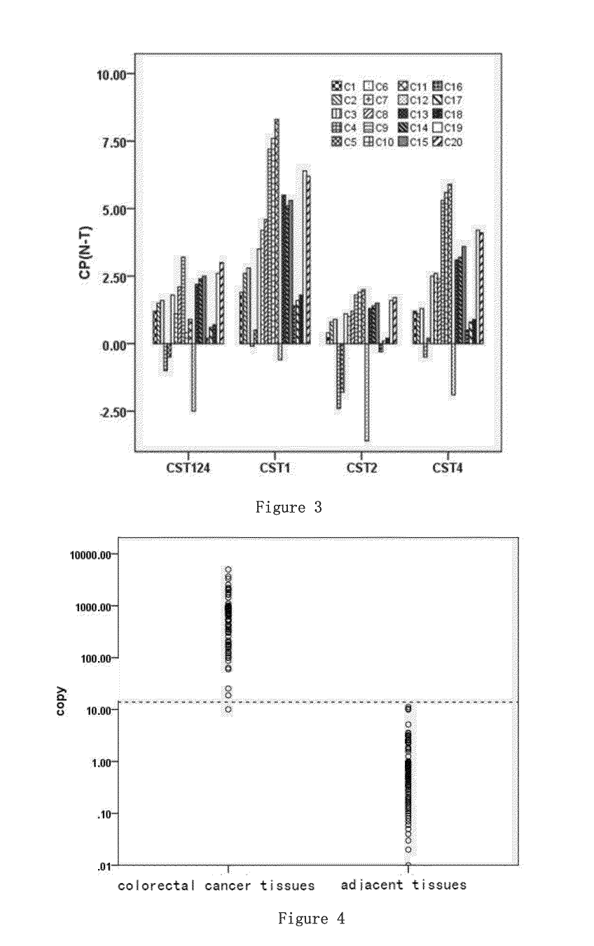

Expression of mRNA of CST4, CST124, CST1 and CST2 in twenty pairs (numbered as C1, C2 . . . C20) of colorectal cancer tumors and their respective adjacent tissues was compared. It was discovered that CST4 mRNA expression difference in tumors and their adjacent tissues is larger than all genes except CST1 (FIG. 3). All samples were diagnosed pathologically with colorectal cancer. Real time PCR using fluorescent dye as the probe was used for the quantification of the genes expression, which was verified by simultaneous amplifications of positive and negative control samples. The results of the controls met the expectation.

2. the Testing Kit Based on Real Time PCR with Fluorescent Dyes as the Probe for mRNA Expression Quantification Contains:

1) Primers for CST4 amplification, whose sequences are shown as following:

(SEQ ID No.4)
Upstream primer: agtacaacaa ggccaccgaa gat
(SEQ ID No. 5)
Downstream primer: agaagcaaga aggaaggagg gag
or
(SEQ ID No. 6)
Upstream primer: tacaacaagg ccaccgaaga tga
(SEQ ID No. 7)
Downstream primer: agaagcaaga aggaaggagg gag
or
(SEQ ID No. 8)
Upstream primer: tgctactcct gatggctacc ctg
(SEQ ID No. 9)
Downstream primer: gtggccttgt tgtactcgct gat
or
(SEQ ID No. 10)
Upstream primer: agtacaacaa ggccaccgaa gat
(SEQ ID No. 11)
Downstream primer: taccaggtct attagaagca agaagga
or
(SEQ ID No. 12)
Upstream primer: tgctactcct gatggctacc ctg
(SEQ ID No. 13)
Downstream primer: catcttcggt ggccttgttg tac
or
(SEQ ID No. 14)
Upstream primer: tgctactcct gatggctacc ctg
(SEQ ID No. 15)
Downstream primer: tactcatctt cggtggcctt gtt
or
(SEQ ID No. 16)
Upstream primer: tgggattatc ctattctcct ccttg
(SEQ ID No. 17)
Downstream primer: ctccagcttt gtgctctgcc tct
or
(SEQ ID No. 18)
Upstream primer: tgctactcct gatggctacc ctg
(SEQ ID No. 19)
Downstream primer: ctcatcttcg gtggccttgt tgt
or
(SEQ ID No. 20)
Upstream primer: tacagtgggt gggagtgggt ggt
(SEQ ID No. 21)
Downstream primer: gagtgggtac agcgtgccct tca

Primers for the amplification of β-actin as an internal reference

(SEQ ID No: 30)
Upstream primer: aagatcattgctcctcctg
(SEQ ID No: 31)
Downstream primer: cgtcatactcctgcttgc

2) Reagents for nucleic acids extractions and reverse transcriptions, SYBR Green fluorescent dye, dNTP, Taq polymerase, RNAse-free water, standard solutions, positive and negative control samples, 10× buffer and magnesium chloride solution.

3. CST4 Expression in Colorectal Cancer Tumors and Tumor Adjacent Tissues.

Testing kits for CST4 mRNA expression based on real time PCR with TaqMan probes. The kit contains the following.

1) Primers and probe:

(SEQ ID No. 1)
Upstream Primer: gctctcaccctcctctcctg
(SEQ ID No. 2)
Downstream Primer: tatcctattctcctccttgg
(SEQ ID No. 3)
Probe: 5′-fam-ctccagctttgtgctctgcctctg-tamra-3′

2) Reagents for nucleic acids extractions and reverse transcriptions, dNTP, Taq polymerase, ribonuclease-free water, standard solutions, positive and negative control samples, recombinant plasmid samples with CST4 gene, 10× buffer and magnesium chloride solution.

All samples were diagnosed with colorectal cancer before RNA extraction and reverse transcription, by which cDNA was obtained. Real time PCR was applied for the quantification of the expression of CST4 in colorectal cancer tumors and their respective adjacent tissues. One hundred samples were tested in this trial.

As shown in FIG. 4, CST4 mRNA expression is specifically high in pathologically diagnosed malignant colorectal cancer tumors and low in their respective adjacent tissues. CST4 expression in tumors is around one hundred fold higher than in adjacent tissues, which indicates that CST4 mRNA is an excellent marker for colorectal cancer. Cancerous tissues can be distinguished from the normal tissues if 13.89 is the cutoff value for CST4 copies. Thus 13.89 is proposed as a reference for the clinical diagnosis of colorectal cancers.

4. CST4 Expression in Colonoscopic Samples from Colorectal Cancer and Enteritis Patients

Colonoscopic samples are of great difference from surgical sample in that the percentage of tumor cells varies. Cancerous tissue is sometimes a very small fraction of the whole colonoscopic sample, or no cancerous tissues are included in the colonoscopic samples. The inventors tested and compared the CST4 expression in 36 colonoscopic samples from colorectal cancer patients and 36 colonoscopic samples from enteritis patients. It was discovered that the median of CST4 expression in cancerous samples was 14.3 fold higher than in enteritis samples. Cancerous tumor can be distinguished from inflammations if 60.5 is the cutoff value, which provides a reference for colorectal cancer diagnosis using colonoscopic samples.

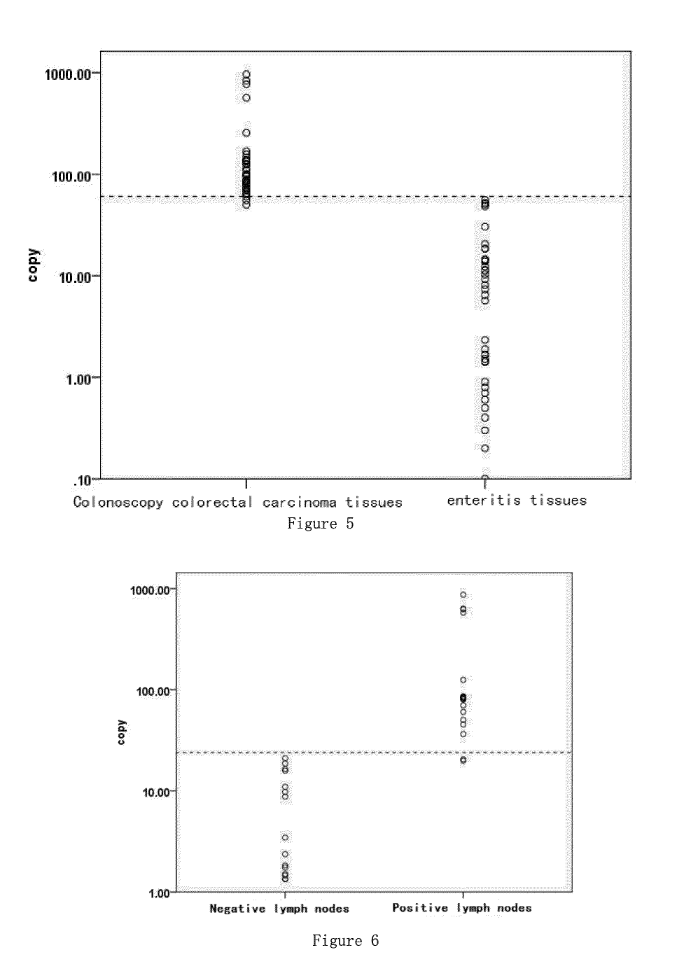

The results are summarized in FIG. 5. Real time PCR was applied for gene expression quantification.

5. CST4 Expression in Lymph Node Sample with and without Colorectal Cancer Metastasis

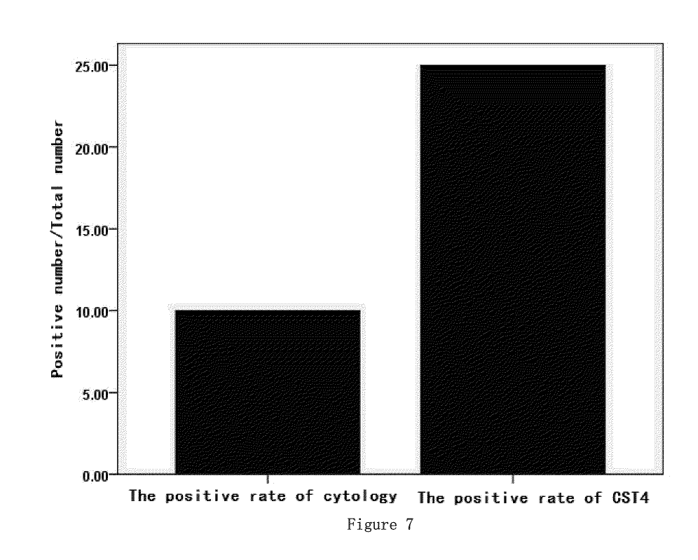

Twenty surgical samples of lymph nodes with pathologically diagnosed colorectal cancerous metastases of varied size and fifteen lymph node samples from patients with early-stage non-metastatic colorectal cancer were obtained. Early-stage cancer patients were selected to avoid undetectable lymph node metastasis and the resulting artifacts. Real time PCR was applied for the quantification of the expressions of CST4 and the detailed experimental procedures were identical as what was described in example 2 As shown in FIG. 6, CST4 expression is high in those samples with colorectal cancer metastasis while it is relatively low in those without cancerous metastasis. Median of CST4 expression in samples with metastasis is 23.6 fold higher than in samples without breast cancer metastasis. It is possible to distinguish cancerous metastasis if the cutoff value is 23.9 copies. Two positive cases of CST4 expression were reported in the non-metastasis group. These samples were carefully studied and micro-metastasis was discovered in both. If these two cases are considered as metastatic samples, CST4 mRNA expression testing is able to distinguish all metastatic cases in the trial. Micro-metastasis which is beyond the capacity of traditional pathological studies was discovered, indicating its higher sensitivity.

6. Accuracy Comparison of CST4 Expression Measurement by Real Time PCR and Cytological Studies for the Detections of Circulating Colorectal Cancer Cells in Peripheral Blood.

RNA was extracted from peripheral blood without red blood cells and platelet; CST4 mRNA expression was quantified by real time PCR, which was compared with samples from enteritis patients and healthy people to decide the existence of circulating colorectal cancer cells. The results were compared with cytological studies.

As summarized in FIG. 7, CST4 expression method is able to detect all cancerous cases diagnosed by cytological studies. Cancer metastasis was discovered in colorectal cancer patients who were diagnosed without metastasis pathologically, evidencing that methods mentioned in the invention are more sensitive than cytological methods; micro-metastasis which is beyond what cytological methods can do can also be detected.

7. Marrow Metastasis Detected by Real Time PCR and Cytological Studies

CST4 mRNA of biopsy marrow samples from colorectal cancer patients was quantified by real time PCR. The results were compared with normal marrow samples for the detections of metastasis or micro-metastasis. The conclusions from these tests were compared with cytological studies.

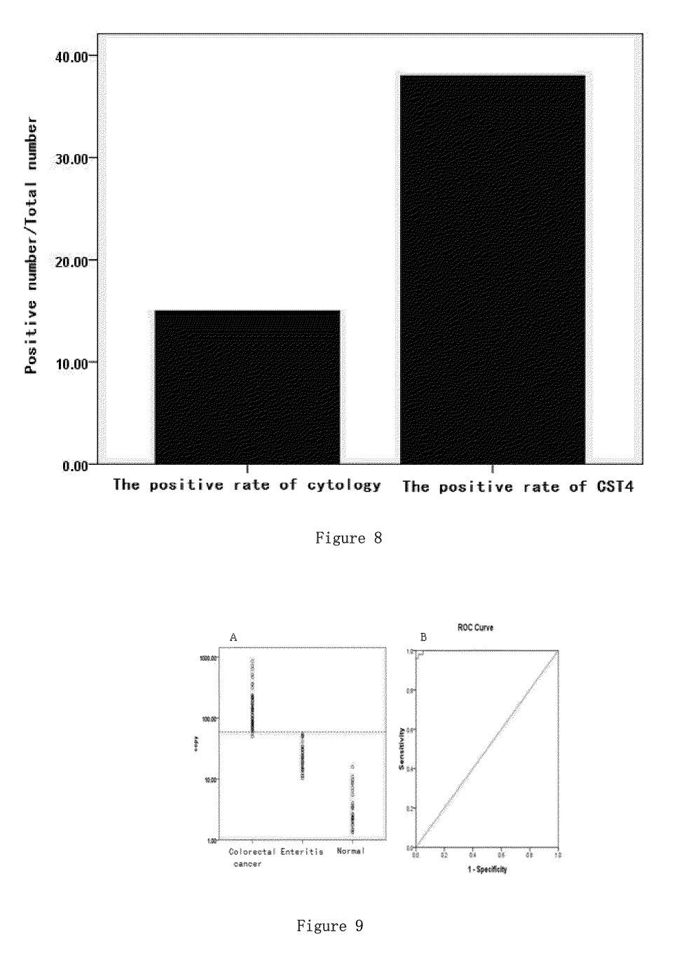

As presented in FIG. 8, 95% samples with marrow metastasis (based on cytological studies) can be detected by CST4 mRNA testing. The higher positive rate than the cytological studies indicates better sensitivity.

8. CST4 Expression in Plasma

9. Cell-Free RNA of Colorectal Cancer Patients, Enteritis Patients and Healthy Subjects

Plasma samples from colorectal cancer patients (50 cases), enteritis patients (30 cases) and healthy subjects (30 cases) were collected. Cell-free RNA was extracted through commercial kits; real time PCR was used for the quantifications of CST4 expression.

It was discovered that median of CST4 expression of the cancerous group is around 6-fold higher than the inflammation group and normal group (FIG. 9A). A cutoff value of 59.48 is able to distinguish the cancerous sample from non-cancerous samples. Receiver operating characteristic (ROC) curve of CST4 expression test as a method for breast cancer diagnosis is presented in FIG. 9B. High sensitivity and specificity are concluded from the curve. CST4 is a specific marker for non-invasive colorectal cancer diagnosis by plasma samples.

10. CST4 Expression in Plasma Cell-Free RNA of Colorectal Cancer Patients, Enteritis Patients and Healthy Subjects Tested by Ligase Chain Reaction (LCR)

The testing kit for CST4 mRNA expression contains the following.

1) Four probes with hapten labeling: gggctctggcctcgagctccaagga (SEQ ID No.33), ataggataatcccaggtggcatctatgatg (SEQ ID No.34), tctcctccttggagctcgaggccagagccc (SEQ ID No.35) catcatagatgccacctgggattatcctat (SEQ ID No.36).

2) Commercially available reagents for nucleic acid extractions and reverse transcriptions. Other reagents are identical to LCx kit (Abbott Laboratories) Cell-free RNA was extracted from plasma samples of colorectal cancer patients (50 cases), enteritis patients (30 cases) and healthy subjects (30 cases). The expression of mRNA of CST4 was tested by LCR method.

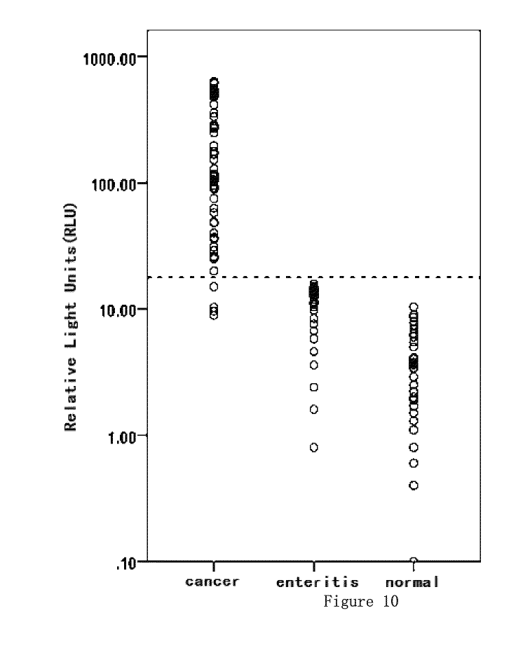

As shown in FIG. 10, the median of the relative light units (RLU) of the cancerous samples is 9.96- and 32.29-fold higher than samples with inflammation and normal samples respectively. Colorectal cancer samples can be distinguished with a cutoff value of 17.85 RLU.

10. CST4 Expression in Serum Cell-Free RNA of Colorectal Cancer Patients, Enteritis Patients and Healthy Subjects Tested by Reverse Transcription Strand Displacement Amplification (rtSDA)

Testing kit for CST4 mRNA expression quantification based on rtSDA contains the following:

1)

(SEQ ID No. 37)
CST4 B1 Primer: cccggcctctgtgtaccctgcta
(SEQ ID No. 38)
CST4 S1 Primer: gaa-ctcgagctaccctggctggggctctgg
(SEQ ID No. 39)
CST4 B2 Primer: ggtggccttgttgtactcgctgat
(SEQ ID No. 40)
CST4 S2 Primer: gct -ctcgag agtgaagggcacgctgtac
(SEQ ID No. 41)
Probe: 5′-³²P-ttactcgag ctccaaggaggagaatagga-3′

2) Reagents for nucleic acid extractions and reverse transcriptions, dCTPaS, dATP, dGTP, dTTP, Bsob I and exo-Bca.

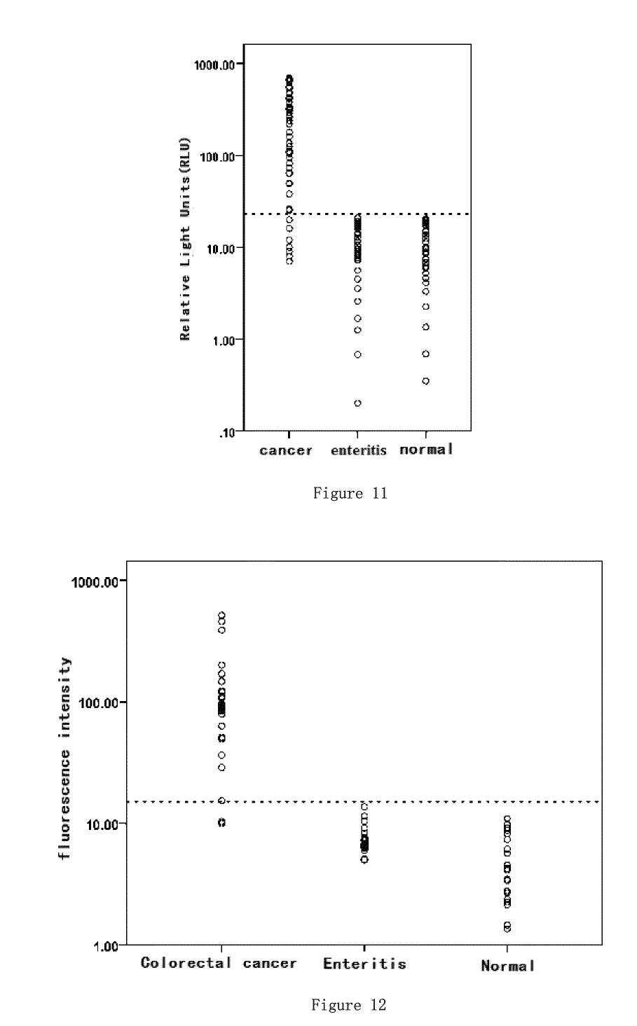

Cell-free RNA was extracted from plasma samples of colorectal cancer patients (50 cases), mastitis patients (30 cases) and healthy people (30 cases). The expression of mRNA of CST4 was tested by thermophilic strand displacement amplification (tSDA). As shown in FIG. 11, the median of the relative light units (RLU) of the cancerous samples is 20.73- and 21.51-fold higher than samples with inflammation and normal samples respectively. Colorectal cancer samples can be distinguished with a cutoff value of 23.09 RLU.

11. CST4 Expression in Urine Cell-Free RNA of Colorectal Cancer Patients, Enteritis Patients and Healthy People

Testing kit for CST4 mRNA expression quantification based on nucleic acid based amplification (NASBA) contains the following.

1) Primers and probes for CST4:

Upstream primer:
(SEQ ID No. 32)
aattctaatacgactcactataggg-gctctcaccctcctctcctg
Downstream primer:
(SEQ ID No. 2)
tatcctattctcctccttgg
Molecular beacon probe:
(SEQ ID NO. 3)

5′-fam-gcggcctccagctttgtgctctgcctctggccgc-dabsyl-
3′

2) Reagents for RNA extraction and reverse transcription, T7 RNA polymerase, RNase H, avian myeloid leukemia virus (AMV) reverse transcriptase, ribonucleotide triphosphate (NTP), deoxyribonucleotide triphosphate (dNTP) and RNA fluorescent dye (Ribo-Green fluorescent dye).

Cell-free RNA was extracted from urine samples of colorectal cancer patients (30 cases), enteritis patients (20 cases) and healthy subjects (20 cases). The expression of mRNA of CST4 was tested by NASBA. As shown in FIG. 12, the median of the relative light units (RLU) of the cancerous samples is about 13.2-fold higher than samples with inflammation and normal samples. Colorectal cancer samples can be distinguished with a cutoff value of 15.02 RLU. CST4 is therefore an excellent marker in non-invasive urine test for colorectal cancer diagnosis

### Example 2

### Applications of CST4 for the Prediction and Diagnosis of Colorectal Cancer

Testing kit for CST4 mRNA expression quantification based on transcription-mediated amplification (TMA) contains the following.

1) Primers and probe for amplification:

Upstream primer:
(SEQ ID No. 32)
aattctaatacgactcactataggg-gctctcaccctcctctcctg
Downstream primer:
(SEQ ID No. 2)
tatcctattctcctccttgg
Molecular beacon probe:
(SEQ ID NO. 3)
5′-fam-gcggcctccagctttgtgctctgcctctggccgc-dabsyl-3′

2) Any reagents included in Gen-probe TMA assay except the primers and probe.

1. CST4 Expression and Colorectal Cancer pTNM Staging

Cell-free RNA in plasma samples from 80 colorectal cancer patients (30 cases of I+II stages and 50 cases of III+IV stages) was extracted by commercial kits. CST4 expression was measured using TMA (transcription-mediated amplification)method.

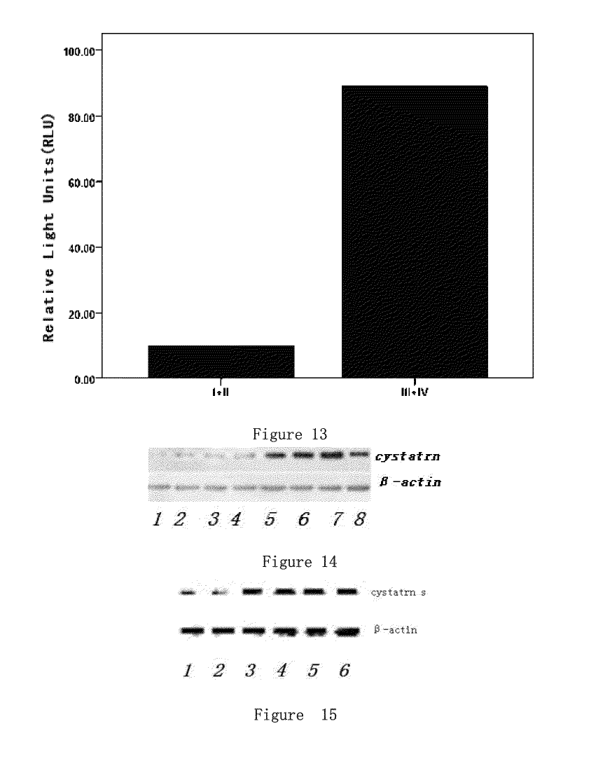

As presented in FIG. 13, RLU median of the late stage group (stages III+IV) is 8.9-fold higher than the early stage group (stages I+II). The results point out that CST4 is a good indicator for colorectal cancer stages and can be used for cancer stage determination.

2. Applications of CST4 Expression in the Real Time Monitoring in Colorectal Cancer Treatment

Serum CST4 expression of colorectal cancer patients taking therapies (6 patients with chemotherapy and 4 patients with radiotherapy) was monitored by real time PCR. The tumor development was compared and correlated with CST4 expression level in blood.

As summarized in Table 1, CST4 expression decreases for patients with effective therapy, which was evidenced by the decreased size of the tumors. CST4 expression increases with the continuation of the therapies for patients with ineffective therapy, which was evidenced by the increased size of the tumors. CST4 is thus proposed as a marker for real time monitoring of therapy efficacy.

TABLE 1
Real time CST4 expression of in colorectal cancer
patients blood during treatment by real time PCR

| | | CST4expression (copy) | | | Tumor Size (cm) | | |
|---|---|---|---|---|---|---|---|
| | | Cycle1 | Cycle2 | Cycle3 | Cycle1 | Cycle2 | Cycle3 |
| Effective | Chemotherapy2 | 781.32 | 521.78 | 80.64 | 2.5 | 1.5 | <1 |
| | Chemotherapy5 | 1533.6 | 1314.6 | 125.25 | 3.5 | 2.5 | <1 |
| | Chemotherapy6 | 1213.5 | 439.89 | 66.64 | 3 | 2 | <1 |
| | Radiotherapy2 | 1434.58 | 1160.33 | 116.83 | 3 | 1.5 | 1 |
| | Radiotherapy3 | 2062.66 | 1689.44 | 189.65 | 3 | 2.5 | 1.5 |
| Ineffective | Chemotherapy1 | 1466.14 | 1984.62 | 2433.57 | 3 | 3 | 3.5 |
| | Chemotherapy3 | 956.92 | 1156.34 | 1846.21 | 2 | 2 | 2.5 |
| | Chemotherapy5 | 646.2 | 826.7 | 1032.55 | 1 | 1 | 1.5 |
| | Radiotherapy1 | 1032.4 | 1246.8 | 1989.61 | 2 | 2.5 | 3 |
| | Radiotherapy4 | 936.4 | 1048 | 1678 | 1 | 1.5 | 2 |

3. CST4 Expression for Colorectal Cancer Prognosis Predictions

The blood CST4 expression of five post-treatment colorectal cancer patients was monitored after 1 month, 3 months and 1 year after the treatment by quantitative real time PCR. As shown in Table 2, two patients have cancerous recurrence. Increasing CST4 expression was observed with these two patients. The cancer recurrences were not detected until the CSTT4 expression reached around 1000 copies. The other three patients did not have cancer recurrence and no significant CST4 expression increase was observed with them. Thus CST4 is a good marker for cancer prognosis predictions.

TABLE 2
CST4 Expression Monitoring of Post-treatment colorectal
Cancer Patients by quantitative real time PCR

| | | CST4 (copy) | | | Tumor Size (cm) | | |
|---|---|---|---|---|---|---|---|
| | | 1 months | 3 monthhs | 1 year | 1 months | 3 monthhs | 1 year |
| Cancer | Patient 1 | 56.84 | 198.87 | 1135.24 | ND | ND | 1 |
| Recurrence | Patient 5 | 15.25 | 64.34 | 786.59 | ND | ND | <1 |
| No Cancer | Patient 2 | 23.6 | 40.5 | 36.3 | ND | ND | ND |
| Recurrence | Patient 3 | 52.43 | 39.8 | 48.65 | ND | ND | ND |
| | Patient 4 | 67.9 | 79.6 | 86.79 | ND | ND | ND |

### Example 3

### Kits for the Prediction and Diagnosis of Colorectal Cancer

1. Testing Kits Based on Taqman Probe for the Real Time Quantification of CST4 mRNA Expression.

1) Primers and probe

(SEQ ID No. 1)
Upstream primer: gctctcaccctcctctcctg
(SEQ ID No. 2)
Downstream primer: tatcctattctcctccttgg
(SEQ ID No. 3)
Probe: 5′-FAM-CTCCAGCTTTGTGCTCTGCCTCTG-TAMRA-3′

2) Reagents for nucleic acids extractions and reverse transcriptions, dNTP, Taq polymerase, RNAse-free water, standard solutions, positive and negative control samples, 10× buffer and magnesium chloride solution.

2. Testing Kits Based on Fluorescent Dye for the Real Time Quantification of CST4 mRNA Expression

1) Primers for CST4

(SEQ ID No. 1)
Upstream primer: gctctcaccctcctctcctg
(SEQ ID No. 2)
Downstream primer: tatcctattctcctccttgg

Primers for β-actin

(SEQ ID No. 30)
Upstream primer: aagatcattgctcctcctg
(SEQ ID No. 31)
Downstream primer: cgtcatactcctgcttgc

2) Reagents for nucleic acids extractions and reverse transcriptions, SYBR Green fluorescent dye, dNTP, Taq polymerase, RNAse-free water, standard solutions, positive and negative control samples, 10× buffer and magnesium chloride solution.

3. Testing Kits Based on Nucleic Acid Based Amplification (NASBA) for the Real Time Quantification of CST4 mRNA Expression

The kit contains

1) Primers and probe

Upstream primer:
(SEQ ID No. 1)
aattctaatacgactcactataggg-gctctcaccctcctctcctg
Downstream primer:
(SEQ ID No. 2)
tatcctattctcctccttgg
Molecular beacon robe:
(SEQ ID No. 3)
5′-FAM-CTCCAGCTTTGTGCTCTGCCTCTG-dabsyl-3′

2) Reagents for RNA extraction and reverse transcription, T7 RNA polymerase, RNase H, avian myeloid leukemia virus (AMV) reverse transcriptase, ribonucleotide triphosphate (NTP), deoxyribonucleotide triphosphate (dNTP) and RNA fluorescent dye (Ribo-Green fluorescent dye).

4. Testing Kits Based on Transcription-Mediated Amplification (TMA) for the Real Time Quantification of CST4 mRNA Expression

The kit contains

1) Primers and probe

Upstream primer:
(SEQ ID No. 1)
aattctaatacgactcactataggg-gctctcaccctcctctcctg
Downstream primer:
(SEQ ID No. 2)
tatcctattctcctccttgg
Molecular beacon robe:
(SEQ ID No. 3)
5′-FAM-CTCCAGCTTTGTGCTCTGCCTCTG-dabsyl-3′

2) Any reagents included in Gen-probe TMA assay except the primers and probe

5. Testing Kits Based on Ligase Chain Reaction (LCR) for the Real Time Quantification of CST4 mRNA Expression

The kit contains

1) Four probes with hapten labeling: gggctctggcctcgagctccaagga (SEQ ID No.33), ataggataatcccaggtggcatctatgatg (SEQ ID No.34), tctcctccttggagctcgaggccagagccc (SEQ ID No.35) catcatagatgccacctgggattatcctat (SEQ ID No.36).

2) Commercially available reagents for nucleic acid extractions and reverse transcriptions. Other reagents are identical to LCx kit (Abbott Laboratories)

6. Testing Kits Based on Thermophilic Strand Displacement Amplification (tSDA) for the Real Time Quantification of CST4 mRNA Expression

The kit contains

1)

(SEQ ID No. 37)
CST4 B1 Primer: cccggcctctgtgtaccctgcta
(SEQ ID No. 38)
CST4 S1 Primer: gaa-ctcgagctaccctggctggggctctgg
(SEQ ID No. 39)
CST4 B2 Primer: ggtggccttgttgtactcgctgat
(SEQ ID No. 40)
CST4 S2 Primer: gct -ctcgag agtgaagggcacgctgtac
(SEQ ID No. 41)
Probe: 5'-³²P-ttactcgag ctccaaggaggagaatagga-3'

3) Reagents for nucleic acid extractions and reverse transcriptions, dCTPaS, dATP, dGTP, dTTP, Bsob I and exo-Bca.

### Part II Protein Detections

This invention is presented by the following examples. It should be noted that these example are for clarification for this invention instead of limiting the applications of this invention. Protocols in Molecular Cloning: A Laboratory Manual (Edited by J. Sambrook et al) were strictly followed for experimental procedures if not noted. Or guidelines from the manufacturers were followed. If not mentioned, percentage and fraction are based on weight.

In this section, the detection of protein biomarkers for colorectal diseases detection is mentioned. Antibodies, testing kits and the protocols are discussed in detail. Their applications for colorectal disease diagnosis and monitoring, and efficacy assessment are described, too.

Recombinant cystatin S protein was purchased from Abnova (0.06 μg/μL, Cat. No. H00001472-P01). Rat-anti-cystatin S monoclonal antibody was purchased from R&D with valence of 1:2000 (Cat. No. MAB1296). Rabbit-anti-cystatin S polyclonal antibody was purchased from Abcam with valence of 1:1800 (Cat. No. ab58515).

This invention provides a method for the determination of colorectal tissue condition and prediction of colorectal cancer recurrence and metastasis. The assessment of the therapies can be realized by methods described in this invention. This method measures at least one protein concentrations in samples provided by the patient. Cystatin S protein and its quantitative or semi-quantitative determination in the sample are of great favor. Although various molecules were reported for protein detection, specific antibodies or their fragments of cystatin S, are preferred in this invention. The methods detection protocols and testing kits can be used for colorectal cancer screening of people without cancerous symptoms.

At least one or more (preferred) antibodies or fragments that bind specifically to at least one epitope of cystatin S are applied in the detection mentioned in this invention. The antibodies can be monoclonal or polyclonal. The preferred monoclonal antibody binds cystatin S with the sequence shown in SEQ ID No.50. The antibody is acquired with cystatin S as immunogen. The immunogen concentration is preferred constant. Therefore, this invention provides a method for cystatin S quantification by the immune-response of the antibodies to the presence of cystatin S.

Subjects in this invention are human beings, wherein the immune-response in the test features antibodies and human peptides. The immune-reactions might be measured by any proper methods, which include but not limited to: ELISA, immuno-blotting or the combination of both. Competitive ELISA and double-antibody sandwich ELISA are preferred in this invention

The monitoring and diagnosis of colorectal tissue conditions and the monitoring of the efficacy of the therapies for colorectal diseases can be quantified by the immune-reaction via the quantification of cystatin S expression.

We claim in this invention all testing kits for the detection of the marker (cystatin S and its epitope herein) in the samples. The condition of the colorectal tissue and colorectal diseases can be diagnosed through the kits. The monitoring of colorectal disease therapies and prediction for cancer recurrence and metastasis are among the applications of the kits, too. Anti-cystatin S antibodies or their fragments should be included in the kits. These antibodies or their fragments bind cystatin S in fluidic samples such as serum. The binding event of the antibody or its fragment and cystatin S should be monitored or detected by a reporting unit.

The reporting unit of the preferred or optimized kit can be antibodies or their fragments with functional labeling. Herein, the reporting unit is preferred as appropriate IgG or IgM antibody. The labels can be and are preferred as enzymes that catalyze reactions with substrate color change such as peroxidase. The labels are preferred to be covalently conjugated to the secondary antibody. Or, the labels can be fluorescent dye.

Testing kit based on ELISA is preferred in this invention.

The ELISA testing kits mentioned are competitive ELISA kit or double-antibody sandwich ELISA kit, whose details are described next. At least one antibody or its fragment are incubated with the analyte. The antibody is a monoclonal antibody and preferred as the monoclonal antibodies mentioned above. Cystatin S protein, which is the immunogen for the production of anti-cystatin S antibody, is conjugated with a microplate (solid substrate herein). The pre-incubated mixture is applied on the ELISA plate; unbound antibodies then bind to the proteins immobilized on the plate. The reporting unit is the immunoglobulin especially IgG and IgM, which is capable of detecting the antibodies on the plate. It should be noted that the antibodies are conjugated with the enzymes or fluorescent labeling for detections.

Another methodology of this invention is immune-blotting, or Western Blot, wherein the proteins in the sample are separated by gel electrophoresis such as PAGE and then transferred to solid substrate such as nitrocellulose membrane. One of the transfer methods is the electro-transfer. The analyte interacts with its specific antibody (monoclonal antibody or its fragment preferred). The immune-reaction can be monitored by appropriate methods such as enzymatic/fluorophore labeled anti-antibodies.

Another preferred methodology in this invention is testing kits based on affinity column. In a typical process, the antibody or its fragment is immobilized on the column, and the sample solution passes the column slowly. The antibodies herein are monoclonal or polyclonal. Antibodies mentioned above are preferred.

The sample solution passes the column; when the analyte protein interacts with the immobilized antibodies, it remains in the column. The analyte protein is then eluted by applying competitive antigen for the antibodies or by changing the running buffer conditions. If multiple proteins are to be analyzed, it is preferred to elute the proteins at different time. The proteins can be quantified by various methods which are well known, such as UV absorbance detection.

Cystatin S testing kits are provided in this invention. Two-channel cystatin S indicator accurately tells whether the cystatin S level is higher than normal or not. Fluidic samples are preferred for the testing. The kit includes containers for the sample, antibodies and their fragments and indicators. Monoclonal antibodies are of favor, especially the antibodies mentioned above. All solutions and buffers necessary for the testing and manuals for operations and data interpretation should be included in the kit.

The kit should be carried on by professionals in any locations such as hospitals, clinics and houses.

Samples to apply this invention include but not limited to serum, plasma, urine and blood, which includes whole blood or its fractions.

As mentioned above, antibodies in this invention can be used for the diagnosis of colorectal cancer and prediction for its recurrence and metastasis. Well known methods in this filed can be combined with the kit. For instance, fluorescence method can be combined with the kit to test the cystatin S level in plasma, serum or urine, and then the existence of a specific disease.

The purpose, advantages and features of this invention are revealed in the following non-limiting examples. Besides, experimental details that support our claims and conclusions made above are also included in the following examples.

### Materials and Methods

Recombinant cystatin S protein was purchased from Abnova (0.06 μg/μL, Cat. No. H00001472-P01).

Antibodies: Rat-anti-cystatin S monoclonal antibody was purchased from R&D with valence of 1:2000 (Cat. No. MAB1296). Rabbit-anti-cystatin S polyclonal antibody was purchased from Abcam with valence of 1:1800 (Cat. No. ab58515).

Immuno-precipitation: 2 mM Phenylmethanesulfonyl fluoride (PMSF), staphylococcal protein A immobilized agarose gel and cystatin S antibody are added in the sample. The mixture is gently stirred under 4° C. overnight. Dimethl pimelimidate is applied to conjugate the anti-cytatin S antibody to the agarose gel. The precipitates are washed and treated with N-glycosidase F and the protein is purified by SDS-PAGE.

In short, Before the application of N-glycosidase F, the precipitate is boiled in 10 μL citrate buffer (50 mM, pH 6.0, 0.5% SDS). 10 μL phosphate buffer (200 mM, pH 8.0 with 40 mM EDTA) and N-octyl glucoside (3%) and N-xylanase are added in the mixture (40 mU), which is incubate overnight (37° C.). Loading buffer are then added and boiled for SDS-PAGE purification. Unless noted elsewhere, 15% polyacrylamide gel is used for PAGE. The gel is imaged by 20% 2,5-diphenyl oxazole solution.

Protein electro-transfer and immune-blotting: Transfer the protein to the nitrocellulose membrane, which is incubated in PBS buffer with 5% skimmed milk power and 0.1% Brij-35 for 2 hours (ambient temperature). The membrane is then incubated in rabbit-anti-cystatin S polyclonal antibody solution overnight (4° C.). The membrane is washed by PBS buffer (with 0.1% Brij-35) for three times. It is incubated with peroxidase labeled goat-anti-rabbit IgG solution (0.27 μL, Jackson) for an hour (37° C.), followed by four times washing by PBS buffer (with 0.1% Brij-35) and one time washing of PBS. The membrane is imaged by commercial TMB solution (TMB Peroxidase Substrate, Kirkegaard and Perry Laboratories Inc. (Gaithersburg, Md.) Cat. No. 50-76-01). Or it's tested by ELC method, during which the membrane is soaked in a solution with 5.4 mM hydrogen peroxide solution, 2.5 nM luminol and 400 mM p-coumaric acid (dissolved in 100 mM Tris-HCl, pH 8.5) and imaged on Agfa CP-BU foil.

Competitive ELISA: ELISA plate (Corning) is coated by cystatin S solution (5 μg/mL) and backfilled by 3% BSA solution. Eight serum samples (2× diluted) and polyclonal rat-anti-cystatin S antibody (with valence of 1:1000) are incubated overnight (4° C.) and applied on the pre-treated ELISA plate. The plate is incubated for one hour under 37° C. Samples holes are washed by TBS buffer (10 mM Tris-HCl, 154 mM NaCl, pH 7.5). Add alkaline phosphatase (ALP) labeled goat-anti-mouse IgG (Jacksonwi ImmunoResearch with valence of 1:2000) solution was added and incubated for an hour (37° C.). TMB solution (TMB Peroxidase Substrate, Kirkegaard and Perry Laboratories Inc. (Gaithersburg, Md.) Cat. No. 50-76-01) is added and OD at 405 nm is quantified by a microplate reader.

Double-antibody Sandwich ELISA: ELISA plate (Corning) is coated by monoclonal rat-anti-cystatin S solution (5 ng/mL) and backfilled by 3% BSA solution. Eight serum samples (2× diluted) are incubated in the holes of the plate for an hour (37° C.). The plate is washed by TBS buffer (10 mM Tris-HCl, 154 mM NaCl, pH 7.5). Biotinylated rabbit-anti-cystatin S polyclonal antibody (valence: 1:1000) is applied in the holes and incubated for an hour (37° C.). Wash the holes with TBS buffer and add streptavidin-peroxidase conjugate (ABC complex). Incubate the plate for 1 hour under 37° C. and wash the holes with TBS buffer. TMB solution (TMB Peroxidase Substrate, Kirkegaard and Perry Laboratories Inc. (Gaithersburg, Md.) Cat. No. 50-76-01) is added and OD at 405 nm is quantified by a microplate reader.

### Example 1

### Detection of Cystatin S

1. Cystatin S Detection in Colorectal Cancer Cell Line Culture Supernatant

CST4 mRNA is over expressed in colorectal cancer tumors as discussed above. As a secretion protein, cystatin S can be found in various body fluids and secretions. In order to establish cystatin S as a marker for colorectal cancer, the supernatant of SW480 and CaCO2 with high expression of CST4 mRNA was loaded on 15% polyacrylamide gel (Lanes 5-6, Lanes 7-8 respectively, FIG. 14); control samples (healthy subjects serum) were loaded on the gel (Lanes 1-2, Lane 3-4, respectively, FIG. 14). After the electrophoresis, the protein was transferred to a nitrocellulose membrane, which reacted with anti-cystatin S antibody and goat-anti-rabbit IgG with peroxidase labeling. TMB was applied for protein imaging. Methods described above were followed.

As shown in FIG. 14, β-actin (internal reference) is at the bottom of the gel. A band with 16 kDa protein was observed in Lanes 5-8 while for Lanes 1-4, the bands were very faint.

2. Cystatin S Detection in Colorectal Cancer Patients Serum

Protocol described in section “1” was followed. As shown in FIG. 15, 3-actin (internal reference) is at the bottom of the gel. A band with 16 kDa protein was observed in Lanes 3-6 (cancerous sample) while for Lanes 1-2 (control sample), the bands were very faint.

3. Serum Cystatin S Level Determination by ELISA Using Monoclonal Antibodies

Experimental: Cystatin S (5 μg/mL) was applied on the ELISA plate, which was incubated overnight (4° C.). Serum samples (30 from colorectal cancer patients and 20 from healthy people) were mixed with anti-cystatin S monoclonal antibody (valence 1:2000, dissolved in TBS with 3% BSA) and incubated overnight (4° C.). The sample mixture was applied on the pre-treated ELISA plate, which was incubated for an hour (ambient temperature). The plate was washed by TBS buffer and incubated with goat-anti-rabbit antibody (0.08 μg/mL, dissolved in TBS). The plate was subjected to reaction with p-nitrophenyl phosphate (p-NPP, CHEMICON International). Microplate reader is used for quantification.

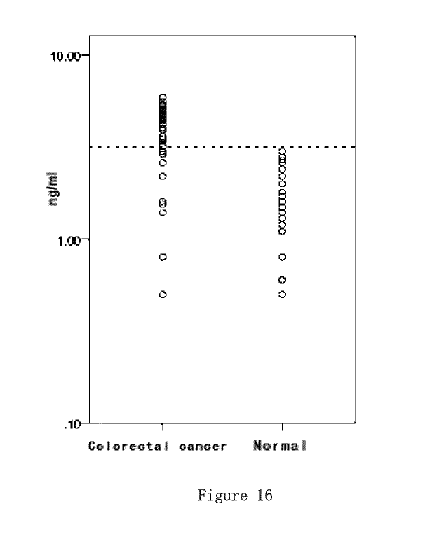

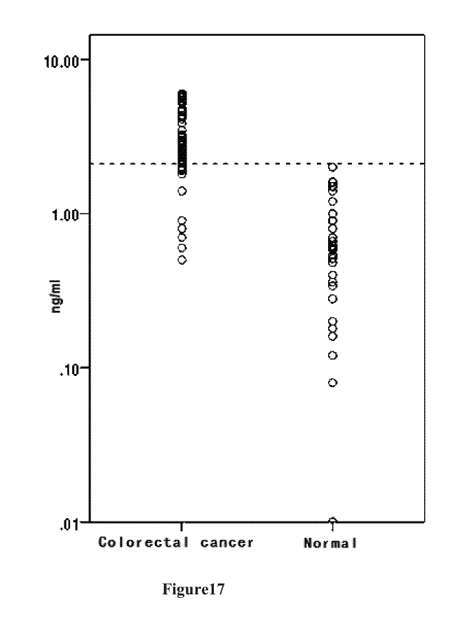

As shown in FIG. 16, the median of cystatin S level in normal serum is 1.55 ng/mL while it is 3.75 ng/mL in colorectal cancerous serum. A cutoff vale of 3.179 ng/mL is capable for the distinguishing of cancerous samples and normal samples.

4. Comparisons of the Sensitivity and Specificity of Cystatin S and CEA for Colorectal Cancer Diagnosis and Prediction

Cystatin S was measured following the method described in section “3”. CEA was measured using commercial kit (DRG, Germany, Cat. No. EIA5071) and user manual is followed.

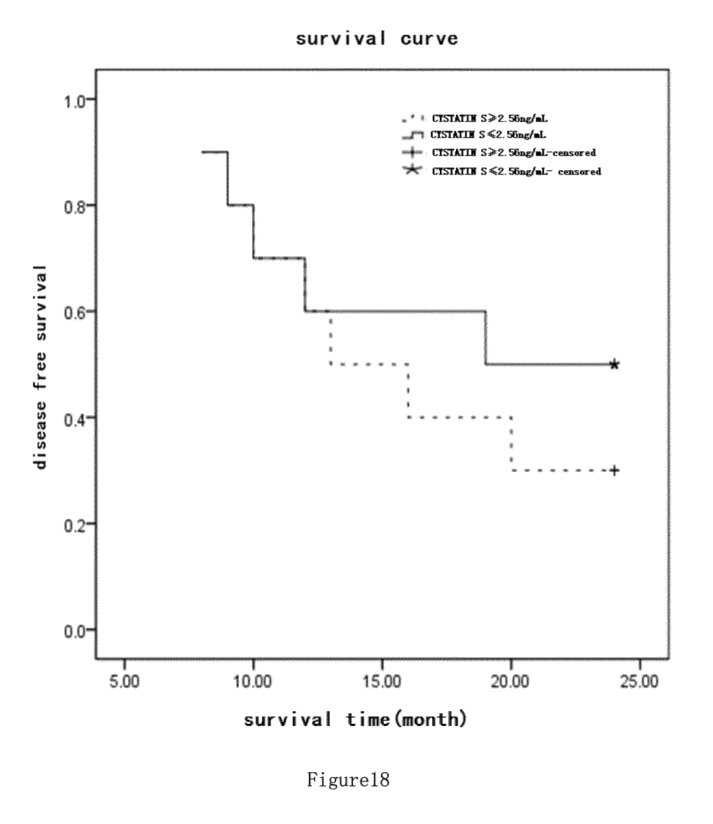

As shown in FIG. 17 and Table 3, the area under curve (AUC) value of the receiving operating characteristic (ROC) curve is 0.698 for cystatin S and 0.573 for CEA, which means the former has better sensitivity and selectivity.

TABLE 3
AUC of the ROC curves

| | | | | Confidence Interval | |
|---|---|---|---|---|---|
| | | | P: comparison of | (cl 95%) | |
| Protein | AUC | Std. Dev. | AUC and 0.5 | Lower | Caps |
| Cystatin S | 0.698 | 0.073 | 0.018 | 0.555 | 0.842 |
| CEA | 0.573 | 0.087 | 0.384 | 0.404 | 0.743 |

5. Methods, Testing Kits and Protocols

Methods, testing kits and protocols that are non-limiting and demonstrative.

Methods for colorectal cancer detection include the following Immobilize cystatin S antigen on a substrate. Apply R&D antibody (MAB 1296) to the pre-treated substrate. Wash the substrate and apply a secondary antibody such as ALP labeled goat-anti-rabbit IgG (Beyotime Inc., Cat. No. A239). Wash the substrate and measure and/or detect the amount of the protein from response from the label (ALP in the case) directly or indirectly.

The substrate include and is not limited to resin particles, cellulose-based materials such as cellulose sheets, plastic plates and particles, etc.

Antigens can be immobilized covalently or non-covalently. Sample for testing is human serum. Substrate selected or preferred should be backfilled by BSA before the addition of the sample to minimize the non-specific interaction between other components in serum and the substrate. The substrate is then washed by proper buffer such as phosphate with surfactant.

A non-limiting example of the labeled secondary antibody is labeled anti-mouse polyclonal antibody. The label include and is not limited to enzymes such as ALP, luciferase, peroxidase, β-galactosidase and fluorescent dyes such as fluorocein. Molecules such as biotin, avidin, streptavidin and digitalis glycoside might be applied for the coupling of the antibody and the label.

If an enzyme is the label, its qualitative and/or quantitative detection can be realized by the addition of the enzymatic substrate and the enzymatic colorimetric and/or luminescent reaction. If a fluorescent dye is the label, its qualitative and/or quantitative detection can be realized by UV exposure and fluorescence measurement/detection. Sensitizer is used if necessary.

As applications of this invention, molecules that bind cystatin S or its epitopes (SEQ ID No.50) are anti-cystatin S antibodies or their fragments, secondary antibodies and solid substrates if necessary, and one or several supporting supplies. These required or optional reagents are provided in the testing kit. The reagents mentioned above might be used for colorectal cancer diagnosis, pTNM stage determination, metastasis detection and evaluation of the therapy efficacy.

For example, the testing kit features specific antibody or its fragment. It also features the reporting unit for the detections of the target protein in the sample optionally or preferably. The reporting unit is preferably a proper secondary antibody, with labeling for detection optionally or preferably (if necessary). The kit optionally or preferably includes one or more buffers such as buffers for protein-substrate incubation and protein backfilling for the removal of the non-specific interactions of proteins in the sample and proteins immobilized on the substrate, as well as washing buffers for the substrate after incubations with sample, secondary antibody and/or reagents mentioned above.

Optionally, the kit provides solid substrate for control protein immobilization for competitive method. In this case, the antibody and the sample are pre-incubated and the mixture is then applied on the substrate. The reporting unit is used for the detection of the antibody and the substrate. Professionals might determine whether the antibody binds the epitope from the serum sample and quantitatively measure the amount of the antibody that binds the serum epitope. Thus the target protein amount can be determined.

Optionally, the kit can be applied in double-antibody sandwich method, wherein anti-cystatin S antibody is immobilized on the substrate. Cystatin S standard solution and pre-treated sample serum are applied on the substrate. Anti-cystatin S polyclonal antibody with reporting-unit-labeling is applied on the substrate. Professionals might determine whether the antibody binds the epitope from the serum sample and quantitatively measure the amount of the antibody that binds the serum epitope. Thus the target protein amount can be determined.

The selection of the testing reagents and/or testing kits, and/or instruments for the measurements and/or requirements for the combinations of the kit and instrument, are dependent upon the methods used for the detection. As mentioned above, these methods include and are not limited to ELISA, protein blotting and flow cytometry. ELISA method are mentioned above and blotting is more accurate but requires more equipment and/or operation time.

6. A Demonstrative Testing Kit and its Protocol

Sample acquisition and storage. For serum, the blood sample is kept for 2 hours under ambient temperature or overnight under 4° C. The sample is centrifuged for 20 minutes (1000×g) and the supernatant is collected as serum. The serum sample should be kept under −20° C. or −80° C. and repeated thawing-freezing should be avoided. For plasma, use EDTA or heparin as the anti-coagulant. Centrifuge the sample for 15 minutes (2-8° C., 1000×g) in less than 30 minutes after blood acquisition. The plasma sample should be kept under −20° C. or −80° C. and repeated thawing-freezing should be avoided. Sample pre-treatment. Serum or plasma samples are recommended to be diluted by 10 times. For instance, mix 100 μL serum or plasma sample with 900 μL PBS buffer. The samples should be diluted by 0.1 M PBS buffer (pH 7.0-7.2)

The testing kit should include the following: 1) ELISA plate closed by a plastic foil; 2) cystatin S standard solutions. Cystatin S solution is prepared with a concentration of 10 ng/mL using PBS buffer with 1% BSA. A series of solutions with concentrations of 5 ng/mL, 2.5 ng/mL, 1 ng/mL and 0.5 ng/mL are prepared by diluting the stock solution. PBS buffer with 1% BSA is used as the solution with 0 ng/mL cystatin S. The solutions should be prepared no more than 15 minutes before the testing. For example, the preparation of 4 ng/mL cystatin S can be realized by mixing 0.5 mL (no less than 0.5 mL) cystatin S solution (8 ng/mL) and 0.5 mL dilution buffer in an Eppendorf tube. Other concentrations can be realized in a similar way. 3) PBST buffer with 3% BSA for backfilling. 4) Antibody for coating: 5 μg/mL rat-anti-cystatin S monoclonal antibody and buffer for dilution (0.05 M NaHCO₃ solution, pH 9.0). 5) Biotinylated rabbit-anti-cystatin S (valence 1:200) and dilution buffer (PBST with 1% BSA). 6) ABC (streptavidin-biotin-peroxidase conjugate). 7) TMB solution for colorimetric detection. 8) PBST buffer (0.05% Tween-20 in PBS). 9) Stop solution: 2 N H₂504.

Detailed operation protocol is described as following.

1) Coating. The rat-anti-cystatin S solution is diluted to 5 μg/mL using sodium bicarbonate buffer (0.05 M, pH 9.0). The solution is applied to the holes (0.1 mL per hole) on a polystyrene plate. The plate is incubated overnight (4° C.). Discard the solution in the holes and wash the holes by washing buffer for three times with three minutes each time.

2) Backfilling. Add 200 μL PBST buffer with 3% BSA in the holes. Incubate the plate for an hour (37° C.) or overnight (4° C.). Discard the solution in the holes and wash the holes by washing buffer for three times with three minutes each time.

3) Sample loading. Assign holes for blank, standard solution and sample solution. Add 100 μL dilution buffer, standard solutions and sample solution to the blank holes, standard holes and sample holes respectively. Avoid bubbles. The solutions should be loaded at the bottom of the holes. Solution contact with hole wall should be avoided. Shake the plate gently and close the holes by a lid or plastic foil. Incubate the plate for 120 minutes (37° C.). Standard solutions should be freshly prepared to ensure the accuracy of the result.

4) Discard the remaining solution in the holes and dry the plate. Do not wash the plate. Add biotinylated rabbit-anti-cystatin S polyclonal antibody solution (200× diluted by PBST with 1% BSA). Close the holes by a lid or plastic foil. Incubate the plate for 60 minutes (37° C.).

5) Incubate the plate for 60 minutes and discard the remaining liquid in the holes. Dry the plate followed by plate washing for 3 times with 1-2 minutes soaking of 300 μL washing buffer each time. Dry the plate or shake the plate to remove the remaining liquid in the holes.

6) Add 100 μL ABC solution to the holes. Close the holes with aa foil and incubate the plate for 60 minutes (37° C.).

7) Incubate the plate for 60 minutes, discard the liquid in the holes and dry the plate. Wash the plate for 5 times with 1-2 minutes soaking of 300 μL washing buffer each time. Dry the plate or shake the plate to remove the remaining liquid in the holes.

8) Successively add 50 μL enzymatic substrate solution in the holes. Close the plate with a foil and incubate the plate in darkness (37° C.) in less than 15 minutes. Stop the reaction when solution in the standard holes has blue color correlated to the concentrations and no color for the blank holes.

9) Stop the reactions by add 50 μL stop solution. The solution turns from blue to yellow. The order for stop solution addition should be identical to that of the enzymatic substrate addition. Stop solution should be immediately added when the reaction is about to the end to ensure the accuracy of the testing.

10) Measure the OD value of the solutions in holes (405 nm), which occurs immediately after the stopping of the enzymatic reactions.

Calculation: OD values versus concentrations of the respective solutions are plotted. The calibration equation is acquired through data fitting and R² is calculated, which should be higher than 0.95 for an effective testing. The analyte concentration is calculated by the OD vale of the sample and the calibration equation.

7. Cystatin S Expression for Colorectal Cancer pTNM Stage Determination

Experimental details are identical to section 6. Samples from 80 cytologically diagnosed colorectal cancer patients (20 T-stage cases, 30 N-stage cases and 30 M-stage cases). As summarized in Table 4, with the development of cancer, cystatin S expression increases, which indicates that cystatin S protein expression can be used for pTNM stage determination.

TABLE 4
Cystatin S expression of colorectal cancer patients with various
pTNM stages

| | | Median of cystatin S expression (ng/mL) |
|---|---|---|
| T-stage | (20 cases) | 0.96 |
| N-stage | (30 cases) | 1.89 |
| M-stage | (30 cases) | 4.04 |

8. Cystatin S Expression for Breast Cancer Metastasis Diagnosis

Experimental details are identical to section 6. Samples from 50 cytologically diagnosed colorectal cancer patients, among which 30 patients have cancer metastasis. As summarized in Table 5, cystatin S expression is higher in those with metastasis than those without cancerous metastasis, indicating that cystatin S expression is a marker for colorectal cancer metastasis diagnosis.

TABLE 5
Cystatin S expression of colorectal cancer patients with and
without metastasis

| | Median of cystatin S expression (ng/mL) |
|---|---|
| No metastasis (20 cases) | 2.43 |
| Metastatic cancer (30 cases) | 3.89 |

9. Cystatin S Expression for the Evaluation of the Endocrine Therapy Combined with Chemotherapy for the Treatment of Colorectal Cancer

Experimental details are identical to section 6. 1000 gastric cancer (NO-1 stages) were studied. The patients were treated with four cycles of 5-FU/LV (levamisole) regimen. All patients were followed up for 24 months. The cystatin S expression was measured after the last cycle of the therapy. 364 cases were validated in the study, whose cystatin S expression median was 2.56 ng/mL. As shown in FIG. 18 disease-free survival (DFS) for patients with higher cystatin S expression than the median was 30%, lower than that of the group with lower cystatin S expression than the median, which was 50%.

It should be noted that all examples describe some feature of the invention for better and clearer presentation. The can be combined in one practice. All features or sub-features can be combined if necessary.

Methods in this invention were illustrated and presented by examples. Many substitutions, modifications and changes are straight forward to professional in this field. Any efforts of these substitutions, modifications and changes are part of the claims and their reasonable extensions. Any publications, patents and patent applications referenced in this patend should be used to illustrate the details for experimental procedures and protocols that are not included in this invention. Any references in mentioned in this invention are not acknowledged as a substitute of the technologies of this invention.

1. The applications of CST4 gene, mRNA of CST4, cDNA of CST4 splices, the corresponding amplicon of CST4-specific primers, cystatin S protein coded by CST4 gene and epitope peptide of cystatin S in the diagnosis and prediction of colorectal cancer. The sequence of CST4 gene is presented in SEQ ID No.42.

2. An application according to claim 1, wherein the sequence of the probe for the detections of CST4 gene, mRNA of CST4, cDNA of CST4 splices is shown in SEQ ID No.3.

3. An application according to claim 1, wherein the specific primers of the amplicon have sequences shown in SEQ ID No.1, 4, 6, 8, 10, 12, 14, 16, 18, 20 (primer 1) and in SEQ ID No.2, 5, 7, 9, 11, 13, 15, 17, 19, 21 (primer 2). Sequence in SEQ ID No.1 pairs with sequence in SEQ ID No.2. Sequence in SEQ ID No.4 pairs with sequence in SEQ ID No.5. Sequence in SEQ ID No.6 pairs with sequence in SEQ ID No.7. Sequence in SEQ ID No.8 pairs with sequence in SEQ ID No.9. Sequence in SEQ ID No.10 pairs with sequence in SEQ ID No.11. Sequence in SEQ ID No.12 pairs with sequence in SEQ ID No.13. Sequence in SEQ ID No.14 pairs with sequence in SEQ ID No.15. Sequence in SEQ ID No.16 pairs with sequence in SEQ ID No.17. Sequence in SEQ ID No.18 pairs with sequence in SEQ ID No.19. Sequence in SEQ ID No.20 pairs with sequence in SEQ ID No.21.

4. An application according to claim 1, wherein the sequence of cystatin S protein's epitope peptide is presented in SEQ ID No.50.

5. An application according to claim 1, wherein diagnosis and prediction refer to the metastasis, micro-metastasis, pTNM stage determination of colorectal cancer, the dynamic monitoring during treatment and tumor progression prediction.

6. Capturers for colorectal cancer markers, wherein the capturers are for biomarkers for colorectal cancer diagnosis and prediction. These biomarkers are CST4 gene, mRNA of CST4, cDNA of CST4 splices, the corresponding amplicon of CST4-specific primers, cystatin S protein coded by CST4 gene and epitope peptide of cystatin S.

7. Capturers according to claim 6, wherein the sequences of the specific primers are presented in SEQ ID No.1-2.

8. Capturers according to claim 6, the sequence of the probe for the detections of CST4 gene, mRNA of CST4, cDNA of CST4 splices is shown in SEQ ID No.3.

9. Capturers according to claim 6, wherein the amplicon's sequence is presented in SEQ ID No. 43.

10. Capturers according to claim 6, wherein these capturers are antibodies that specifically recognize cystatin S or its epitope.

11. Capturers according to claim 6, wherein the sequence of the epitope peptide of cystatin S is shown in SEQ ID No.50.

12. The applications of these capturers in the preparations of testing reagents and kits for colorectal cancer detection.

13. Diagnostic kits that include capturers according to claim 6.

14. Diagnostic kits according to claim 13, wherein these kit shall be 1) testing kits for quantitative and real time detection of the mRNA of CST4 based on hydrolytic Taqman probe. The primers sequences are presented in SEQ ID No.1-2; the probe sequence is presented in SEQ ID No.3. 2) Testing kits for quantitative and real time detection of the mRNA of CST4 based on fluorescent dye. The primers sequences are presented in SEQ ID No.1-2. Sequences for internal calibration primers are shown in SEQ ID No.30-31. 3) Testing kits for quantitative and real time detection of the mRNA of CST4 based on nucleic acid based amplification (NASBA) or transcription-median amplification (TMA). Both kits include primers and probes for CST4, whose sequences are shown in SEQ ID No.2, 32 (for primers) and 3 (for probe). 4) Testing kits for quantitative and real time detection of the mRNA of CST4 based on ligase chain reaction (LCR). Four probes are included whose sequences are shown in SEQ ID No.33-36. 5) Testing kits for quantitative and real time detection of the mRNA of CST4 based on thermophilic strand displacement amplification (tSDA). Primers (sequences shown in SEQ ID No.37-40) and a probe (SEQ ID No. 41) are included.

15. Testing kits according to claim 13, wherein the detailed descriptions are as following: 1) Double-antibody sandwich ELISA kits, including the solid substrate, capturers immobilized on the solid substrate, biotinylated capturers and the enzymatic substrate (colorimetric). Capturers immobilized are monoclonal antibodies while biotinylated capturers are polyclonal antibodies. Or 2) Blotting kits including solid substrate, capturers, enzymatic labeled secondary antibody and enzymatic substrate for colorimetric detections. The capturers are monoclonal antibodies and biotinylated capturers are polyclonal antibodies. Or 3) Competitive ELISA kits including solid substrate, immobilized antigen, biotinylated capturers, the enzymatic substrate for colorimetric detections and specific monoclonal antibody. The biotinylated capturers are polyclonal antibodies.

16. Testing kits according to claim 14, wherein positive and negative controls and blank samples are included.

17. Double-antibody ELISA testing kits according to claim 15, wherein the monoclonal antibody is rat-anti-cystatin S antibody; the solid substrate is ELISA plate and the biotinylated polyclonal antibody is biotinylated rabbit-anti-cystatin S polyclonal antibody.

18. Double-antibody ELISA testing kits according to claim 15, wherein the solid substrate is ELISA plate, wherein the immobilized capturer is rat-anti-cystatin S antibody, wherein the biotinylated capturer is rabbit-anti-cystatin S polyclonal antibody, wherein the biotinylated capturer is rabbit-anti-cystatin S polyclonal antibody (with valence of 1:1000) and wherein the substrate for colorimetric detection is alkaline phosphate (ALP).
Or the kit is based on competitive ELISA, wherein the ELISA plate is the solid substrate, wherein the concentration of cystatin S is 5 μg/mL, wherein the specific monoclonal antibody is rat-anti-cystatin S antibody (with valence of 1:2000), wherein enzymatic labeled secondary antibody is ALP-labeled goat-anti-mouse IgG (with valence of 1:2000) and wherein the substrate for colorimetric detection is ALP substrate. The volume ratio of cystatin S, enzymatic labeled secondary antibody and ALP substrate is 1:2.
Or the kit is based on immunoblotting, wherein the sold substrate is nitrocellulose membrane, wherein the capturer is monoclonal cystatin S antibody (with valence of 1:1000), wherein the enzymatic labeled secondary antibody is peroxidase labeled goat-anti-rabbit IgG and wherein the enzymatic substrate is TMB solution.

19. Protocols of the testing kits according to claim 17, wherein the details are described as following: Coat the ELISA plate by rat-anti-cystatin S antibody, which is backfilled by 3% BSA afterwards. Apply samples with eight-fold dilution to the plate and incubate it under 37° C. Wash the holes with samples by TBS and add biotinylated rabbit-anti-cystatin S polyclonal antibody. Incubate the plate under 37° C. Wash the holes with samples by TBS and add streptavidin-biotin-horseraddish peroxidase (HRP) complex. Incubate the plate under 37° C., followed by plate washing by TBS. Finally, the analyte is quantified by the addition of alkaline phosphatase (ALP) and reading of QD (405 nm) on a microplate reader.

20. Indicative for the diagnosis of cancer and a kit, wherein the kit for the detection of protein levels CYSTATIN S, including solid-phase carrier, immobilized on a solid phase support the capture agent, the biotinylated capture agents chromogenic substrate; immobilized on a solid phase carrier-specific monoclonal antibody as the capture agent, the capture agent that specifically biotinylated polyclonal antibody;
Or the kit for the detection of CYSTATIN S protein level, the kit is a solid phase carrier CYSTATIN S protein, CYSTATIN S specific murine monoclonal antibody, HRP secondary antibody and chromogenic substrates, including solid-phase carrier bag thereof;
The kit for detecting or CYSTATIN S protein levels, including solid-phase support, the capture agent and HRP-chromogenic substrate, the capture agent comprises a specific monoclonal antibody, the capture agent that specifically biotinylated multi-resistant.
